(19) United States
(12) Patent Application Publication
(10) Pub. No.: US 2009/0142355 A1
(43) Pub. Date: Jun. 4, 2009

(54) DIAGNOSIS AND TREATMENT OF TYPE 1 DIABETES

(76) Inventor: Chester A. Alper, Brookline, MA (US)

Correspondence Address: RISSMAN JOBSE HENDRICKS & OLIVERIO, LLP, 100 Cambridge Street, Suite 2101, BOSTON, MA, 02114, US

(21) Appl. No.: 12/112,576

(22) Filed: Apr. 30, 2008

Related U.S. Application Data
(60) Provisional application No. 60/926,955, filed on Apr. 30, 2007.

(51) Int. Cl. A61K 39/395; C12Q 1/68; A61K 31/7088; C40B 30/00

(52) U.S. Cl. 424/152.1; 435/6; 514/44; 506/7

(57) ABSTRACT

Methods for the diagnosis and treatment of type 1 diabetes are described. HLA-DQA2 has now been identified as the gene which is primarily responsible for determining whether an individual is susceptible to develop type 1 diabetes. The use of HLA-DQA2 as a target for developing therapeutic and diagnostic targets for treating type 1 diabetes and detecting susceptibility for the disease is disclosed.

### CROSS-REFERENCE TO RELATED APPLICATIONS

This application is based on and claims the benefit of U.S. Provisional Application No. 60/926,955, filed Apr. 30, 2007, the disclosure of which is incorporated by reference herein in its entirety.

### GOVERNMENT SPONSORED RESEARCH OR DEVELOPMENT

This work may have been funded in whole or in part by a grant from the federal government. The federal government may have certain rights in the invention.

### BACKGROUND OF THE INVENTION

This invention relates to the diagnosis and treatment of type 1 diabetes (“T1D”). More specifically, the invention relates to the detection of elevated levels of the transcription and/or the translation of the gene HLA-DQA2 as a diagnostic tool for the onset of type 1 diabetes. The invention also relates to the inhibition or disruption of HLA-DQA2 protein production for the treatment of type 1 diabetes. The invention further relates to targets for the development of therapeutic tools for the treatment and diagnosis of this disease.

Type 1 diabetes (“T1D”), or insulin-dependent diabetes mellitus (“IDDM”) is an autoimmune disease associated with the selective destruction of pancreatic B cells and chronic insulin deficiency that affects 1 in 300 people in the U.S. It has been known for decades that there is a genetic basis for T1D. It is highly likely that one of the genes involved is in the major histocompatibility complex (“MHC”), but the precise identification of the MHC susceptibility gene(s) involved has proved elusive. The association of T1D with certain HLA genes, notably HLA-DRB1*0301, -DQB1*0201 (DR3), and HLA-DRB1*04, -DQB1*0302 (DR4, DQ8), is generally recognized by the increased frequency in Caucasian T1D patients compared with matched controls, and may mark susceptibility. Other MHC haplotypes are decreased in frequency among patients, particularly HLA-DRB1*1501, -DQB 1*0602 (DR2, DQ6) and HLA-DRB 1*04, -DQB1*0301 (DR4, DQ7), and are considered protective. Non-HLA-DR2, -DR3 and -DR4 haplotypes mark lesser degrees of susceptibility or protection and some, such as HLA-DR1, are neutral, with similar frequencies in patients and controls.

MHC associations may reflect a susceptibility gene in the HLA region of chromosome 6 (IDDM1), demonstrated by the higher concordance rate of T1D in HLA-identical siblings of patients (about 15%) than siblings in general (about 6%). The fact that monozygotic twin (“MZT”) disease concordance, 30-50%, is even higher than that in HLA-identical sibs indicates that other, unlinked susceptibility genes must also be involved in T1D pathogenesis (i.e., T1D is polygenic). The MZT concordance rate suggests that only 30-50% of all genetically susceptible unrelated persons in the general population actually have T1D (i.e., there is incomplete penetrance of susceptibility genes).

Family studies indicate that the known MHC markers for both susceptibility to and protection from T1D are parts of large (1-4 Mb or longer) fixed stretches of DNA called conserved extended haplotypes (“CEHs”). CEHs account for about 50% of all normal European Caucasian MHC haplotypes and for about 65% of European Caucasian T1D patient haplotypes. The identity of DNA on independent examples of individual CEHs is supported by evidence of identical alleles. Recently, it has been shown that there is at least 99% identity of single nucleotide polymorphisms (SNPs) over 2.9 Mb of the HLA-B8, DR3 CEH. Similarly, at least 99% conservation of SNPs over 4 Mb on the HLA-B18, DR3CEH has been demonstrated. In general, patients carry the complete CEHs (not just the markers). This hinders the identification of the MHC T1D susceptibility gene. Therefore, approaches that study genes on protective HLA-DR, -DQ haplotypes or on non-CEHs in T1D patients may be the most likely to be informative for susceptibility gene localization and identification.

Incidents of type 1 diabetes are rising at the rate of about 3% to 5% per year. As effective interventions become available, knowing the true susceptibility gene becomes increasingly urgent. Accordingly, it is essential to know which gene is the type 1 diabetes susceptibility gene, not only to understand the pathogenesis of the disease, but also to devise optimal tests for high-risk subjects to determine who will ultimately develop disease, and to develop therapeutic approaches for treating the disease.

The human major histocompatibility complex (“MHC”) on chromosome 6p has been studied intensively, inter alia, in connection with diabetes. It is of great interest because it contains highly polymorphic genes that determine immune function, the susceptibility to a remarkably large number of complex diseases, and the outcome of tissue transplantation. The MHC genes that have been the focus of attention include class I (including, among others, HLA-A, HLA-Cw and HLA-B) that occupy over 1 Mb of genomic DNA and produce similar single surface-expressed 45 kD polypeptide alpha chains that combine with non-MHC-encoded beta chains (beta₂-microglobulin). The intermediate (or class III region) is about 1 Mb in size, and its genes produce a heterogeneous group of molecules, many of which are secreted, such as TNF a and the complement proteins C2, factor B, C4A and C4B. Finally, the most centromeric class II region includes HLA-DRA, -DRB1, -DRB3, -DRB4 and -DRB5, and HLA-DQA1-DQB1 and -DPA1, -DPB1, encoding surface-expressed α and B chains that form heterodimeric HLA-DR, HLA-DQ and HLA-DP molecules, which is also about 1 Mb in size.

Many class I and class II MHC molecules are involved in the binding of antigenic peptides and their presentation to T cells in the adaptive immune response. The class II region also includes less well-characterized genes, including HLA-DQB3, HLA-DQA2, HLA-DO, HLA-DM and HLA-DN, some of which may not be expressed.

The mode of inheritance of the MHC type 1 diabetes susceptibility gene is important to the design of definitive experiments to determine its identity. The type 1 diabetes MHC susceptibility gene is recessive as previously suggested when it was noted that MHC haplotype sharing by T1D-affected sib pairs was most consistent with that mode of inheritance. Formal analyses of MHC haplotype sharing by affected sib pairs supports this conclusion.

Another approach to defining the mode of inheritance of the type 1 diabetes MHC susceptibility gene is the fit of HLA-DR genotypes in patients of to the Hardy-Weinberg equilibrium (“HWE”), since if the MHC susceptibility gene is recessive, the distribution of HLA-DR3/DR3, HLA-DR3/DR4, and HLA-DR4/DR4 genotypes should fit the Hardy-Weinberg equilibrium. Surprisingly, in many populations of Caucasian type 1 diabetes patients to the Hardy-Weinberg equilibrium (“HWE”), since if the MHC susceptibility gene is recessive, the distribution of HLA-DR3/DRS, HLA-DR3/DR4, and HLA-DR4/DR4 genotypes should fit the Hardy-Weinberg equilibrium. Surprisingly, in many populations of Caucasian type 1 diabetes patients, there is an excess of HLA-DR3/DR4 heterozygotes, and a paucity of HLA-DR3/DR3 and HLA-DR4/DR4 homozygotes. However, patients of some ethnic groups have no significant deviation from the Harvey-Weinberg equilibrium. Moreover, the distribution of homozygotes and heterozgotes fit the Harvey-Weinberg equilibrium. These findings suggest that, although the MHC susceptibility gene is recessive, the distribution of HLA-DR genotypes may often be distorted by population effects.

It is therefore an objective of this invention to identify the gene or nucleic acid segment which is responsible for type 1 diabetes susceptibility in humans. It is also an objective of this invention to provide methods for diagnosing type 1 diabetes in humans. It is further objective of this invention to provide methods for developing therapeutic treatments for type 1 diabetes, and the therapeutic treatments developed thereby.

### SUMMARY OF THE INVENTION

According to the present invention, it has now been discovered that HLA-DQA2 (major histocompatiliabilty complex, class II, DQ alpha 2) is a marker for type 1 diabetes susceptibility. This marker is used herein to develop diagnostic methods for predicting the onset of type 1 diabetes and therapeutic methods for the treatment of this disease.

In one embodiment, the invention is a diagnostic method for determining whether an individual will develop type 1 diabetes. The diagnostic method comprises measuring the level of transcription or translation of HLA-DQA2 for a patient, and comparing this measured level against a predetermined level for a normal subject. An increase in the level of transcription or translation above a predetermined level is a positive indication of the propensity of an individual for developing type 1 diabetes. Preferably, the level of protein expressed by HLA-DQA2 is measured, preferably by an antibody to the protein, and used as a diagnostic indicator for the disease.

In another embodiment, the invention is a therapeutic treatment for type 1 diabetes which comprises administering to a patient in need of treatment an effective amount of an agent for inhibiting HLA-DQA2 transcription or translation. Preferably, the agent comprises an antibody, such as a monoclonal antibody, to the protein encoded by HLA-DQA2 which resides on a B cell in a patient. Alternatively, the agent interferes with or blocks the transcription or translation of HLA-DQA2, and is an siRNA molecule. The invention further comprises pharmaceutical compositions formulated from such agents, and other adjuvants and excipients.

In a further embodiment, the invention is a target for identifying and selecting therapeutic and diagnostic agents for treating and diagnosing type 1 diabetes comprising HLA-DQA2, HLA-DQA2 fragments, or B cells containing the protein expressed by HLA-DQA2. This embodiment comprises, for example, contacting an agent of interest with B cells expressing the HLA-DQA2 protein, and determining the response of the agent on the ability of the B cells to express the protein. High-throughput screening assays can advantageously be used to identify active candidates from a library of potential biologically active agents.

The various features and advantages of the present invention will be better understood from the following specification when read in conjunction with the accompanying drawings.

### BRIEF DESCRIPTION OF THE DRAWINGS

FIGS. 1A and 1B are graphs illustrating the predicted frequencies for recessive (FIG. 1A) and dominant (FIG. 1B) inheritance of an MHC disease susceptibility gene at various susceptibility gene frequencies. Observed haplotype distribution frequencies for multiple sclerosis (“MS”) and T1D are marked on the graphs. Although MS frequencies fit either mode of inheritance, only recessive inheritance fits for T1D.

FIG. 2 are a series of graphs showing the distribution of CEHs and fragments on HLA-DR2, -DR3 and DR4 haplotypes.

FIGS. 3A and 3B are graphs showing immunological deficiencies in homozygotes (open bars), heterozgotes (gray bars) and non-carriers (black bars) for the following CEHs: HLA-B8, SC01 and DR3 (FIG. 3A) and HLA-B18, F1C30 and DR3 (FIG. 3B).

FIG. 4 is a graph showing HLA-DQA2 transcription determined by qRT-PCR in a total of 13 random normal controls and 10 T1D patients. As controls, RT-PCR determined relative transcription of HLA-DQA1 in the same 13 controls, and 10 T1D patients showed variation but no mean difference between T1D patients and controls (not shown).

### DETAILED DESCRIPTION OF THE INVENTION

The invention provides method for detecting the onset of type 1 diabetes, and methods for the treatment of this disease. The invention is based on the discovery that the HLA-DQA2 gene is primarily responsible for susceptibility of individuals to contracting type 1 diabetes as described herein.

As used herein, the following terms and phrases shall have the following meanings unless indicated otherwise.

A “subject” or “patient”, as used herein, includes mammals such as human and non-human mammals. Veterinary applications are deemed within the scope of the present invention.

The abbreviation “T1D” as used herein designates type 1 diabetes.

The terms “treatment” or “treating” a medical condition, such as type 1 diabetes, are intended to include both prophylactic and therapeutic methods of treating a subject. “Treatment” generally denotes the administration of a therapeutic agent to a subject having a disease or disorder, a symptom of a disease or disorder, or a predisposition toward a disease or disorder, for the purpose of preventing, alleviating, relieving, reducing the symptoms of, altering, or improving the medical condition or disorder. The methods of treatment described herein may be specifically modified or tailored based on a specific knowledge of the subject obtained by pharmacogenomics, and other methods for analyzing individual drug responses to therapies.

An “inhibitor” in the context of the invention generally denotes an agent that can inhibit the transcription or translation of the HLA-DQA2 gene. By “inhibiting” an interaction is generally meant, in the context of this invention, that the transcription or translation of the HLA-DQA2 gene is interrupted. Such inhibition can result from a variety of events, such as by interrupting, preventing or reducing the transcription or translation, inactivating the protein produced by the gene, such as by cleavage or other modification, preventing or reducing the expression of the protein on a cell, expressing an abnormal or inactive protein, deactivating the protein, preventing or reducing the proper conformational folding of the protein, interfering with signals that are required to activate or deactivate the gene, or interfering with other molecules required for the normal synthesis or functioning of the gene. Examples of types of inhibitors useful in the present invention are inhibitory proteins, such as antibodies, inhibitory peptides and proteins, inhibitory carbohydrates, inhibitory glycoproteins, chemical entities, and small molecules.

In particular, inhibitory proteins include antibodies to the HLA-DQA2 protein, including humanized antibodies, chimeric antibodies, Fab₂ antibody fragments, polyclonal antibodies, and monoclonal antibodies. Monoclonal antibodies are preferred.

Inhibitory peptides include peptides or fragments that recognize the binding site, or a portion of the binding site, of the HLA-DQA2 protein to the B cell such that the binding of the protein to the B cell is reduced or eliminated.

Chemical entities and small molecules which are designed to interrupt the transcription or translation of HLA-DQA2 are also within the scope of the inhibition.

A “therapeutically effective amount” of a pharmaceutical composition means that amount which is capable of treating, or at least partially preventing or reversing the symptoms of, the medical condition or disease state. A therapeutically effective amount can be determined on an individual basis and is based, at least in part, on a consideration of the particular species of mammal, for example, the mammal's size, the particular inhibitor used, the type of delivery system used, and the time of administration relative to the progression of the disease. A therapeutically effective amount can be determined by one of ordinary skill in the art by employing such factors and using no more than routine experimentation.

The inhibitors of this invention can be incorporated into pharmaceutical compositions suitable for administration to a subject. Such compositions typically comprise the inhibitor and a pharmaceutically acceptable carrier. As used herein, the language “pharmaceutically acceptable carrier” is intended to include any and all solvents, dispersion media, coatings, antibacterial and antifingal agents, isotonic and absorption delaying agents, and the like, compatible with pharmaceutical administration. The use of such media, and agents for pharmaceutically active substances is well known in the art. Except insofar as any conventional media or agent is incompatible with the active compound, the use thereof in the pharmaceutical compositions of the invention is contemplated. Supplementary active compounds can also be incorporated into the present compositions.

The administration of the active compounds (inhibitors) of the invention may be for either a prophylactic or therapeutic purpose. Accordingly, in one embodiment, a “therapeutically effective dose” refers to that amount of inhibitor sufficient to result in a detectable change in the physiology of a recipient patient. In another embodiment, a “therapeutically effective dose” refers to an amount of inhibitor sufficient to result in modulation of HLA-DQA2 expression. In yet another embodiment, a “therapeutically effective dose” refers to an amount of inhibitor sufficient to result in the amelioration of symptoms of type 1 diabetes. In still another embodiment, a “therapeutically effective dose” refers to an amount of inhibitor sufficient to prevent the occurrence of type 1 diabetes in a patient.

Toxicity and therapeutic efficiency of the inhibitory compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of the affected tissue, i.e. the bone marrow in most instances, in order to minimize potential damage to uninfected cells, and thereby to reduce side effects.

Data obtained from cell culture assays and animal studies can be used in formulating a range of dosages for use in humans. The dosage of such compounds lies preferably within a range of circulation concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Generally, the therapeutically effective amount of the pharmaceutical compositions used herein will vary with the age of the subject and condition, as well as the nature and extent of the disease, all of which can be determined by one of ordinary skill in the art. The dosage may be adjusted by the physician, particularly in the event of any complication. A therapeutically effective amount will typically vary from 0.01 mg/kg to about 1000 mg/kg, preferably from about 0.1 mg/kg to about 200 mg/kg, and most preferably from about 0.2 m/kg to about 20 mg/kg.

The present invention encompasses active agents which modulate or inhibit HLA-DQA2 transcription or translation. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds having a molecular weight less than about 10,000 grams per mole, organic or inorganic compounds (i.e., including heterorganic and organometallic compounds) having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecules depend upon a number of factors within the knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of execution, any drug combination, and the degree of expression or activity to be modulated.

In certain embodiments of the invention, a modulator or inhibitor of HLA-DQA2 activity is administered in combination with other agents (e.g., a small molecule), or in conjunction with another, complementary treatment regime for type 1 diabetes.

The pharmaceutical compositions can be included in a container, pack, or dispenser together with instructions for administration.

The pharmaceutical compositions of the invention can include any pharmaceutically acceptable carrier known in the art. Further, the composition can include any adjuvant known in the art, e.g., Freund's complete or incomplete adjuvant. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcohol/aqueous solutions, emulsions or suspensions, including saline and buffered media. Parenteral vehicles include sodium chloride solution, Ringer's dextrose, xylitol, dextrose and sodium chloride, lactated Ringer's solution or fixed oils. Intravenous vehicles include fluid and nutrient replenishers, electrolyte replenishers (such as those based on Ringer's dextrose or xylitol), and the like. Preservatives and other additives may also be present such as, for example, antimicrobials, antioxidants, chelating agents, inert gases and the like.

The pharmaceutical compositions can be administered to the mammal by any method which allows the inhibitor to reach the appropriate B cells. These methods include, e.g., injection, infusion, deposition, implantation, oral ingestion, topical administration, or any combination thereof. Injections can be, e.g., by intravenous, intramuscular, intradermal, subcutaneous or intraperitoneal administration. Single or multiple doses can be administered over a given time period, depending upon the progression of the disease, as can be determined by one skilled in the art without undue experimentation. Administration can be alone or in combination with other therapeutic agents. The route of administration will depend on the composition of a particular therapeutic preparation of the invention, and on the intended site of action. The present compositions can be delivered directly to the site of action.

Other delivery systems can include time-release, delayed release or sustained release delivery systems. Such systems can avoid repeated administrations of the active compounds of the invention, thereby increasing the convenience to the subject and the physician. Many types of delayed release delivery systems are available and known to those of ordinary skill in the art. These include polymer-based systems such as polylactic and polyglycolic acid, polyanhydrides and polycaprolactone; nonpolymer systems include lipids such as sterols, and particularly cholesterol, cholesterol esters and fatty acids or neutral fats such as mono-, di and triglycerides; hydrogel release systems; silastic systems; peptide based system, was coatings, compressed tablets using conventional binders and excipients, partially fused implants and the like. In addition, pump-based hardware delivery systems can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Pharmacogenomics thereby allows a clinician or physician to target prophylactic or therapeutic treatments to patients who will most benefit from the treatment and to avoid treatment of patients who will experience toxic drug-related side effects.

Although it has previously been widely believed that HLA-DQB1 is the MHC T1D susceptibility gene, it is likely that it is merely a good marker for T1D rather than the susceptibility gene itself. Cogent evidence against HLA-DQB1 as the susceptibility gene is the occurrence of protective HLA-DQB1*0602 alleles with normal nucleotide sequences in some patients. The most reasonable interpretation is that these haplotypes carry susceptibility alleles at the true susceptibility locus.

Another reason that HLA-DQB1 (or HLA-DRB1, DQB1) may not be the susceptibility gene(s) is that many HLA-DRB1, DQB1 haplotypes are either neutral or only weakly susceptibility-conferring or protective. For the true susceptibility locus, alleles should be either susceptibility-conferring or protective.

One possible reason for the existence of weak or neutral markers may be that 50% or more of MHC haplotypes in normal Caucasians, and 65% or more in patients with T1D, are conserved extended haplotypes that have fixed DNA over 1 to more than 4 Mb of DNA. On the better marker haplotypes (HLA-DRB1*1501, DQB1*0602; HLA-DRB1*0301, DQB1*0201; HLA-DRB1*04, DQB1*0302; and HLA-DRB1*04, DQB1*0301), the DNA fixity includes the susceptibility locus. On other haplotypes (neutral or weakly protective or susceptibility-conferring such as those with DR1, DR5, or DR8), this fixity is lacking and there has been ancient crossing over so that there is either no difference in the ratio of susceptibility-conferring to protective alleles at the true susceptibility locus, or only a weak preponderance of susceptibility-conferring or protective alleles. Thus, HLA-DR, DQ and HLA-DQB1 are good markers for but are not themselves believed to be the true susceptibility loci for T1D.

Spurred by the completion of the sequencing of the human genome some years ago, statistical methods of disease susceptibility gene localization were introduced, based on SNPs. SNPs occur on average every 290 bp of genomic DNA, and form haplotypes that are deduced from studies of the DNA of many individuals by linage disequilibrium (LD) analysis. These haplotypes that are deduced from studies of the DNA of many individuals by linkage disequilibrium (LD) analysis. These haplotypes identify islands of nucleotide sequence stability (blocks) between which meiotic crossing over occurs (and occurred historically) as “hotspots.” The blocks are operationally defined by strong LD (D′ near 1) between the outermost defining SNP pairs and a greater than 19-fold ratio of strong to weak LD (D′ significantly <1) among all internal pairwise comparisons. Using this definition, these studies have determined that the vast majority of such blocks are between 5 and about 200 kb in size, although the possibility of occasional larger blocks (up to 804 kb) has also been suggested. Not stressed, but sometimes found, is LD between blocks that may define stretches of fixity of up to 500 kb on 38% of chromosomes. Remarkably, a relatively small number (2 to 4) of SNP hapoltype variants account for 80%-95% of all observed chromosome and are found in all human populations studied, although relative frequencies vary. Thus, the results of SNP-LD analysis suggest that the human genome (including the MHC) consist of 5 to 200 kb blocks between which random recombination occurs.

However, when applied to the MHC, the SNP-LD analysis in particular, and any statistical approach in general, cannot detect the kind of fixity over long stretches of DNA (CEHs) or the extreme polymorphism of many MHC alleles and haplotypes. Although the method performs better if there is prior knowledge of CEHs, it is far less informative than direct haplotype definition in families.

The pedigree analysis and a direct observations approach can be used to determine population MHC haplotypes composition. One simply counts the number of observed similar haplotypes (identical or near-identical for HLA-B, complotype, HLA-DR/DQ specificities or alleles) in any particular group of families to define statistically significant differences in MHC haplotype frequencies in populations characterized by ethnicity or by the presence of a patient (association studies), or to determine whether DNA conservation extends to intervening or nearby polymorphic loci. For a disease, the haplotypes composition of chromosomes occurring in patients in the families is compared with the haplotype composition of all other chromosome occurring in those families, thus providing ethnically-matched family control haplotypes. This principle is the same as that of the more recently introduced transmission distortion test, but provides more information. For determining significantly different allele or haplotypes frequencies, simple X² statistics suffice, including for showing that the fixity of these similar haplotypes is statistically significant by LD analysis.

Direct determination of MHC haplotypes from family study shows similar-sized small blocks as those detected by SNP-LD analysis, but otherwise gives a very different and more complex picture. At least half of Caucasian haplotypes are fixed from HLA-B to HLA-DR/DQ (˜1 Mb) as CEHs, some of which extend over 3-4 Mb or more. The prediction that independent examples of any given CEH would have the same or nearly the same nucleotide sequence, has been borne out by the demonstration of identical alleles and functions encoded by independent examples of a number of CEHs.

Evidence has been obtained for >99% identify of SNPs over 4.5 Mb of over 2 dozen independent examples of the CEH [HLA-B18, F1C30, DR3], from HLA-A through HLA-DPB1, in Basque patients with T1D or celiac disease. Fixity of >2.9 Mb has been shown for the [HLA-B8, SC01, DR3] CEH. These fixed haplotypes differ in frequency in different Caucasian subpopulations and in Caucasian patients with different HLA-associated diseases, complicating disease susceptibility gene localization. If the marker for a disease is a 3-4 Mb piece of DNA rather than single MHC alleles, no information as to which gene on the haplotypes is the susceptibility gene is obtainable if the patients mainly carry the intact CEH.

Despite the fact that is has been known that the MHC contains a susceptibility gene (or genes) for T1D, the specific locus (loci) and responsible alleles have not been identified. Although it is widely believed that HLA-DQB1 is the locus (or that alleles of the HLA-DRB1, -DQA1, -DQB1 block together confer susceptibility or protection), a brief review of available facts suggests that this is not so and that alleles at this locus (loci) are only good marker of the unknown true susceptibility locus. Assuming recessive inheritance of the true T1D MHC susceptibility gene(s), the following facts support the view that HLA-DRB1, -DQB1 provide good markers for T1D but are not themselves the true susceptibility loci:

1. The striking markers for susceptibility to T1D in Caucasians, HLA-DR3, DQ2 and HLA-DR4, -DQ8 are parts of a limited set of CEHs: [HLA-B8, SC01, DR3, DQ2]; [HLA-B18, F1C30, DR3, DQ2]; [HLA-B62, SC33, DR4, DQ8]; [HLA-B38, SC21, DR4, DQ8]; [HLA-B62, SB42, DR4, DQ8]; and [HLA-B60, SC31, DR4, DQ8]. Recent dense SNP analyses showed that fixity usually extends centromerically beyond HLA-DQB1 on the examples of these CEHs that have been studied.
2. Although similar information is not yet available for the protective CEHs [HLA-B7, SC31, DR2, DQB1*0602], [HLA-B44, SC30, DR4, DQ7], [HLA-B44, FC32, DR7, DQ2] and [HLA-B57, SC61, DR7, DQ9], it is reasonable to assume that the region of conservation on normal haplotypes usually extends beyond HLA-DQB1 to include “protective” alleles of the true susceptibility locus.
3. Protective alleles or haplotypes (HLA-DR2 or HLA-DRB1*1501, -DQB1*0602, for example) are sometimes found in T1D patients.
4. The same HLA-DQB1 alleles that are protective in one population may mark susceptibility in another.
5. HLA-DQB1 alleles that occur in different haplotypes with different HLA-DRB1 alleles have different relative risks or odds ratios.
6. Many HLA-DQB1 alleles or HLA-DRB1, -DQB1 haplotypes are neutral or only mildly protection- or susceptibility-conferring. This has never been explained.
HLA-DR2 haplotypes, in general, have frequencies of 0.04 and 0.15 in populations of T1D patients and controls, respectively. In particular, a subset of HLA-DR2 (HLA-DRB1*1501, -DQB1*0602) is relatively rare among European Caucasian T1D patients at perhaps 0.01 to 0.02 frequency vs. around 0.07 in controls. In one patient with the [HLA-B7, SC31, Dr2] CEH, instead of the protective HLA-DQB1*0602, the susceptibility allele HLA-DQB1*0402 was found. This is consistent with an ancient meiotic crossover between HLA-DRB1 and HLA-DQB1 with HLADQB1 (or a locus centromeric to HLA-DQB1) being the T1D susceptibility locus. But, in 14 T1D patients who carried HLA-DRB1*1501, -DQB1*0602, the HLA-DQB1*0602 genes had normal nucleotide sequences. This is consistent with the susceptibility gene being centromeric to HLA-DQB1.

HLA-DQB1 57 non-asp has been suggested as a T1D susceptibility determinant. T h is postulate has many problems. The neutral HLA-DRB1*01, -DQB1*0501 encodes HLA-DQB1 57 non-asp. Japenese T1D patients are often HLA-DQB1 57 asp homozygous and some Caucasian T1D patients carry DQB1 57 asp. The frequency of DQB1 57 asp was around 0.075 in the Basque T1D population. Moreover, in the Basques, no HLA-DRB1*04, DQB1*0301 (DQB1 57 asp) occurred among controls, although there were 3 examples among T1D patients. All of these findings point to the marked population dependence of the phenomenon. Finally, the whole HLA-DQB1 57 asp question is moot if HLA-DQB1 is not the susceptibility locus. Thus, there are many reasons why alleles of HLA-DQB1 or HLA-DRB1, -DQB1 are good markers for, but are not themselves, the susceptibility locus (loci).

There are a number of open reading frames as well as recognized genes between HLA-DQB1 and HLA-DPB1: HLA-DQB3, -DQA2, -DQB2, -DOB, TAP-2, LMP-7, TAP-1, LMP-2 and -DOA. The literature on HLA-DQB3, HLA-DQA2 and HLA-DQB2 is controversial. HLA-DQA2 has been variously described as non-polymorphic, polymorphic, non-expressed and expressed in B cells and B cell lines. At first intron HLA-DQA2 Taq I RFLP was described and claimed as a good T1D marker. It was stated that the Taq I site is on the HLA-DRB1*04, DQB1*0302 (DR4, DQ8) haplotype.

Initial reports found no expression of HLA-DQA2, but the gene was later found to be transcribed and translated constitutively in B cells. Although there are no reported non-synonymous nucleotide differences in the exons, the HLA-DQA2 gene has a 3.4 kb first intron that shows extensive polymorphism (49 SNPs, excluding DR4, DQ8) and has 19 SNPs in the 5′ UT region among the 4 haplotypes (B8, DR3; B7, DR2; B18, DR3; and B62, DR4, DQ7) already fully sequenced by the Sanger Centre. The 388 bp 5′ UT region on HLA-B8, DR3 has been shown to have identical sequences in T1D patients and normal subjects. This was also true of the 5′ UT region, SNPs in HLA-DQA2 on DR4, DQ8 haplotypes. This would appear to rule out HLA-DQA2 as a T1D susceptibility locus. It is believed that this simply demonstrates that the fixity of DNA extends through HLA-DQA2 on these haplotypes in both patients and controls, as would be expected whether HLA-DQA2 or a locus centromeric to it is the susceptibility locus. HLA-DQB3 and HLA-DQB2 are less controversial. They appear not to be expressed in normal B cells.

It has been known for decades that only 30 to 50% of MZT of a T1D patient also have the disease despite having presumably identical genes. From the first observation of this phenomenon, differing environmental triggering events have been considered to be responsible for the discrepancy. A favorite environmental factor has been viral infection, with rubella and Coxsakie viruses among the more popular candidates. Despite intense study, no environmental factor has been convincingly demonstrated. Recent large-scale studies of twins have revealed that the incidence of T1D is identical in dizygotic twins and siblings, making any different environmental trigger unlikely. A similar identity of rates in dizygotic twins and sibs was found for the antibody markers of impending T1D. A possible role for an epigenetic mechanism affecting MHC gene expression for incomplete penetrance has been suggested.

There is the possibility in T1D of a situation analogous to that affecting HLA-B27 in AS. Specific variants of HLA-B27 have been considered to be the susceptibility genes for AS. HLA-B27 expression is significantly higher in HLA-B27-positive AS patients than in healthy HLA-B27-positive normal subjects. Moreover, HLA-B27 protein in patients, in contrast to that of normal subjects. Moreover, HLA-B27 protein in patients, in contrast to that of normal HLA-B27, forms homodimers rather than the usual heterodimers with β2 m, as demonstrated by SDS polyacrylamide gel electrophoresis (with and without reducing agent) and western blotting. These homodimers are functional and appear to be involved in interaction with NK cells and CD8+ and (remarkably) CD4+ T cells. It is not clear how these abnormalities arise or are involved in the pathogenesis or are the result of AS.

Increasing attention has been paid recently to allele-specific gene expression, particularly with respect to changes in methylatoin and its relation to complex disease. Allele-specific regulation of gene expression has been demonstrated for HLA-DQA1. Allele-specific regulation of at least 2 putative non-MHC susceptibility genes for T1D has been demonstrated: INS and CTLA4. However, there would appear to be no report of variable (probably acquired), allele-related upregulation or its relation to disease, except for AS.

A model has been developed for understanding the MCH genetics of T1D based on (a) establishing the mode of inheritance of the T1D MHC susceptibility gene, (b) taking into account variable conservation of stretches of DNA in the human genome (CEHs and their fragments) and how this relates to MHC markers for T1D susceptibility, (c) deriving the aggregate frequency of susceptibility alleles from MHC haplotypes sharing by affected sib pairs and (d) exploring the basis of penetrance, using CEHs and a prospective method, and showing that penetrance is an inherent MHC gene-related process.

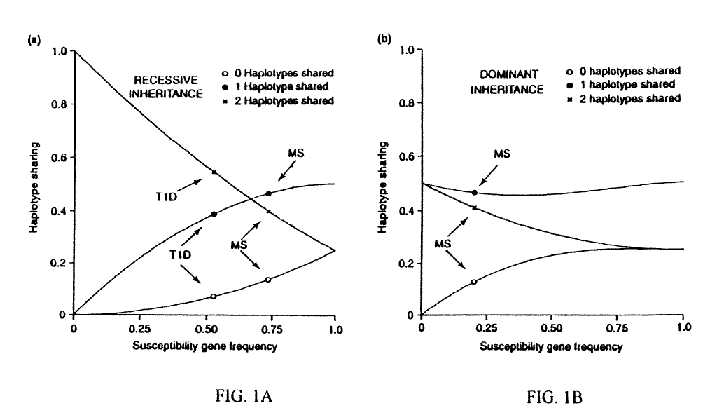

A method for calculating the MHC susceptibility gene frequency for MHC-associated polygenic disease from published MHC haplotypes sharing in affected sib pairs of 55% for 2 MHC haplotypes, 38% for 1 and 7% for none is used. The method avoids an incorrect Bayesian “correction” of a fixed (not statistical) determination used in the literature in the frequency of 0.37+0.08. This analysis (FIG. 1) is consistent with a recessive MHC susceptibility gene for T1D in Caucasians with a frequency of 0.525.

The distribution of homozygotes, heterozgotes and non-carriers of the MHC markers BF*F1 among T1D patients fits the HWE, consistent with recessive inheritance of the MHC susceptibility gene for T1D. The deviations from the HWE of MHC allele genotypes (e.g., HLA-DR3/DR4 over DR3/DR3 or DR4/DR4) in T1D patients that have been reported in many populations requires explanation. One approach is the study of MHC haploptypes in a presumably homogeneous T1D population, the Basques of Bilbao, Spain. Since the Basques use ancestral names for themselves, only families are studied in which all eight such names were Basque. HLA-DRB 1, DQB1 genotypes fit the expectations of the HWE. Thus, as with 2 previously reported populations, MHC genotype distribution is consistent with Mendelian recessive inheritance. To explain the HLA-DR3/DR4 excess among T1D patients in many Caucasian populations over the predictions of recessive inheritance, a theoretical basis for such a phenomenon based on the polygenic nature of the disease is provided. It is hypothesized that, if parents originated from previously isolated Caucasian subpopulations that had selected against alleles at different critical susceptibility loci for a polygenic disease, their offspring could have a greater risk of that disease than either parent had individually. Evidence for this stratification among parents of patients with T1D shows that:

1. Parents transmitting HLA-DR3 to HLA-DR3/DR4 patients have significantly different non-transmitted (non-diabetic) HLA-A allele frequencies from HLA-DR4-transmitters (p<0.025).
2. HLALA-DR3-positive parents of patients have significantly different insulin (INS) gene allele frequencies than HLA-DR4-positive parents (p<0.05).
3. Parent pairs of patients have significantly greater (54% vs. 27%) self-reported ethnicity disparity than parent pairs in control families (p<0.001).
4. HLA-DR genotypes of parents of patients did not fit the HWE. Although there are excess HLA-DR3/DR4 heterozygoes among T1D patients, there are significantly fewer HLA-DR3/DR4 heterozyous parents of patients than expected (p<0.001).

These findings are consistent with HLA-DR3 and HLA-DR4 specificities and INS VNTR alleles marking both disease susceptibility and separate Caucasian subpopulations of parents. This hypothesis thus explains a number of previously reported, seemingly disconnected puzzling phenomena: (1) the exess of HLA-DR3/DR4 heterozygotes among patients, (2) the rising world-wide incidence of type 1 diabetes, (3) the reported changing frequency of HLA-DR3/DR4 heterozygotes and of MHC susceptibility alleles in general in patients over the past several decades, and (4) the association of INS alleles with specific HLA-DR alleles in T1D patients. It is believed that the MHC T1D susceptibility gene is inherited as a simple recessive, often complicated and distorted by parental subpopulation stratification.

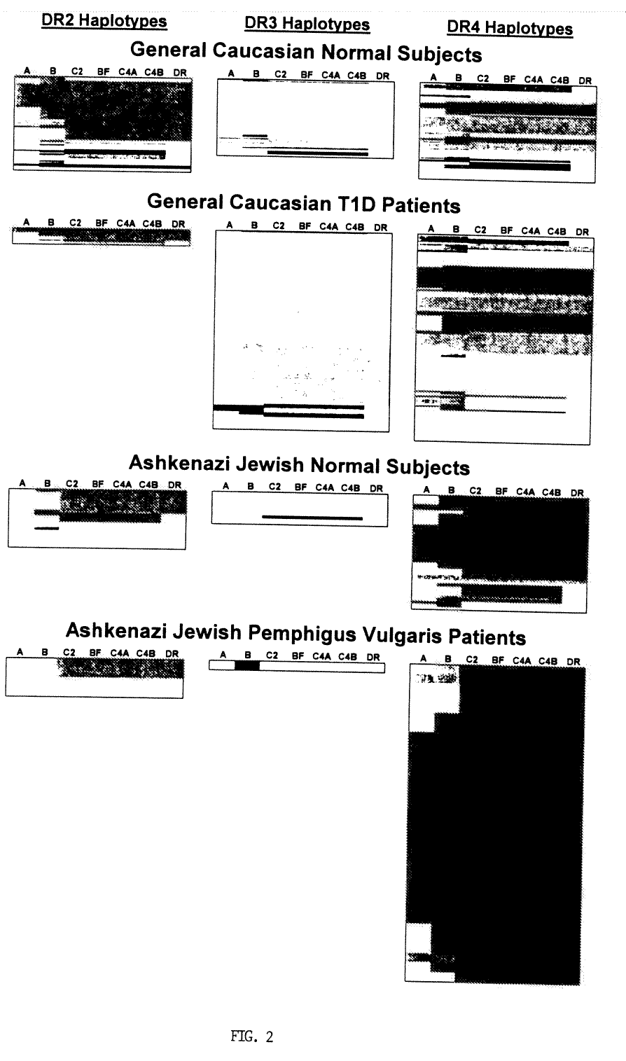

TABLE 1
HLA-DR2-, -DR3, or DR4-containing CEHs with a frequency of 0.02 or higher in
at least 1 of the specified (FIG. 2) populations of Caucasian haplotypes.

| | | | T1D | | | |
|---|---|---|---|---|---|---|
| | | Gen | Gen | Ashk | PV Ashk | |
| HLA-A^{a} | CEH | Cauc^{b} | Cauc | Jewish | Jewish | HLA alleles |
| A3, | B7, SC31, | 0.069 | 0.015 | 0.005 | — | HLA-A*0301/0201, B*0702, |
| | DR2 | | | | | |
| A1 | B8, SC01, | 0.086 | 0.183 | 0.036 | — | HLA-A*0101,B*0801, |
| | DR3 | | | | | DRB1* |
| A30 | B18, F1C30, | 0.006 | 0.077 | 0.005 | — | HLA-A*3001, B*1801, |
| | | | | | | DRB1* |
| A26 | B38, SC21 | 0.012 | 0.041 | 0.133 | 0.543 | HLA-A*2601, B*3801, |
| | | | | | | DRB1* |
| A2 | B44, SC30, | 0.026 | 0.020 | 0.010 | — | HLA-A*0201, B*4402, |
| | | | | | | DRB1* |
| A2 | B62, SC33, | 0.010 | 0.044 | 0.005 | — | HLA-A*0201, B*1501, |
| | | | | | | DRB1* |
| A31 | B60, SC31 | 0.009 | 0.035 | — | — | HLA-A*3101, B-4001, |
| | | | | | | DRB1* |
| A2 | B62, SB42, | 0.005 | 0.029 | — | — | HLA-A*0201, B*1501, |
| | | | | | | DRB1* |

^{a}HLA-A specificities shown have a frequency of at least 0.22 on independent examples of the indicated CEHs among the normal haplotypes.
^{b}Frequency of common CEHs among 2000 normal family control Caucasian haplotypes.

The analysis of fragments of CEHs is used to provide some susceptibility gene localization information. Through the examination via pedigree analysis of the enrichment among patients of fragments or blocks of marker CEHs, it has been possible to identify roughly regions within the MHC likely to carry susceptibility genes. In this way, the class II (HLA-DR, DRQ) region has been implicated in pemphigus vulgaris, myasthenia gravis, gluten-sensitive enteropathy and asthma in ragweed allergy and the complotype region in dermatitis herpetiformis. Similarly, studies in IgAd have suggested susceptibility genes in both the class II and class I regions. Because of the rarity of historical recombination within these blocks (even on some non-CEHs), including HLA-DRB1, -DQB1, more precise identification of susceptibility genes has been impeded. Moreover, because marker haplotypes are common in both disease and control haplotypes populations, ancient crossovers in patients may often be between susceptibility CEHs, providing no information.

FIG. 2 (key is in Table 1) shows the distribution of common CEHs (frequency in any group ≧0.02) on HLA-DR2, -DR3 and -DR4 haplotypes among 2000 normal Caucasian, 353 Caucasian T1D, 195 Ashkenazi Jewish normal and 61 Ashkenazi Jewish pemphigus vulgaris (PV) haplotypes. FIG. 2 dramatically illustrates several points. The first is that CEHs represent significant proportions of normal haplotypes. The second is that CEHs in normal haplotypes differ in frequency in different ethnicities. The third is that increases in HLA DR haplotypes among patients are accompanied by dramatic increases in certain CEHs. Finally, the relatively common disease T1D has many CEH markers, whereas the rare (and MHC dominant) disease PV essentially has a single CEH marker (in this population). Finally, there appears to be some enrichment in the complotype-HLA-DR (DQ) region of haplotypes in both diseases (with some variation in HLA-B), but in T1D there is an increase in DR4 haplotypes without other markers of the marker CEHs. All of the HLA-DR4 increase in PV is made up of the single marker CEH.

Preliminary experiments are conducted to define fixity centromeric to HLA-DQB1 on CEHs conferring susceptibility to or protection from T1D. DNA from homozygotes for [HLA-B8, SC01, DR3] and (HLA-DRB1*04, DQB1*0302) (known susceptibility haplotypes for T1D) and for [HLA-B7, SC31, DR2] and (HLA-DRB1*04, DQB1*0301) (known protective haplotypes for T1D) are studied. To evaluate MHC polymorphism centromeric to HLA-DQB1, overlapping long-range PCR and a series of restriction enzymes are used to show a complete lack of polymorphism in multiple examples of each CEH and at least 2 alleles in each of the open reading frames over the 41 kb segment centromeric to HLA-DQB1 differentiating these CEHs and little polymorphism and no open reading frames in the remaining 125 kb of genomic DNA to HLA-DOB. PCR amplicon size varied between 3.7 to 6.1 kb. The (DRB1*04, DQB1*0301) and (DRB1*04, DRQB1*0302) haplotypes differed strikingly in this region from each other and from HLA-DR3 and DR2 haplotypes. Other preliminary evidence includes: (a) in a study of 36 independent instances of the (FIC30, DRB1*0301, DQB1*0201) T1D susceptibility haplotypes in Basque T1D patients and 6 instances of the same haplotype in Basque controls, 86% and 67%, respectively, carries HLA-DPB1*0202, indicating that the great majority of such haplotypes in patients and controls are fixed at least through HLA-DPB1, (b) using 2360 SNPs, DNA fixity on over 4.9 Mb of the [HLA-B18, FIC30, DR3] and [HLA-B98, SC0, DR3] CEHs in 16 Basque T1D patients and 25 of their haploidentical unaffected siblings is demonstrated. In summary, there is fixity beyond HLA-DQB1 on marker susceptibility-conferring and protective CEHs (but presumably not on non-marker haplotypes). Thus, it is the DNA fixity of HLA-DR2, -DQ6; HLA-DR3, -DQ2; HLA-DR4, -DQ7 and HLA-DR4, -DQ8 haplotypes (particularly CEHs) that includes the centromeric true susceptibility locus (probably HLA-DQA2) that makes them good markers for T1D susceptibility.

In general, CEHs are well-characterized telomeric (but not centromeric) to HLA-DQB1. Because protective haplotypes sometimes occur in T1D patients, it is hypothesized that the recessive MHC susceptibility locus is centromeric to HLA-DQB1. This predicts that all instances of protective HLA-DRB1, -DQB1 haplotypes (HLA-DRB1*1501, -DQB1*0602; HLA-DRB1*04, -DQB1*0301; DRB1*07, -DQB1*0202 and DRB1*07, -DQB1*0303) in T1D patients have ancient (in a previous generation) crossing over centromeric to HLA-DQB1 but telomeric to the susceptibility locus. These haplotypes therefore carry susceptibility-conferring alleles at the true susceptibility locus. In T1D patients who carried the protective HLA-DQB1*0602, S alleles and no P allele centromeric to HLA-DQB1 are found, consistent with the true T1D susceptibility gene being centromeric to this locus (see Table 3).

Special care is taken to ensure that HLA-DQA2 and not HLA-DQA1 are analyzed, given the genes high degree of homology. Of the 54 SNPs of the HLA-DQA2 first intron (including on HLA-DR4, DQ8), only 5 differentiated HLA-B7, -DR2 from HLA-DR4, -DQ8. The study involves a directly determined first HLA-DQA2 intron 5-SNP informative haplotype on susceptibility CEHs in patients and normals to define susceptibility alleles S1 and S2 and on protective CEHs in normals to define protective alleles P1 and P2 (see Table 2). There was a single SNP (1217) that distinguishes both S from both P alleles.

TABLE 2
Five-SNP haplotypes that may distinguish susceptibility from protective HLA-DQA2 alleles.

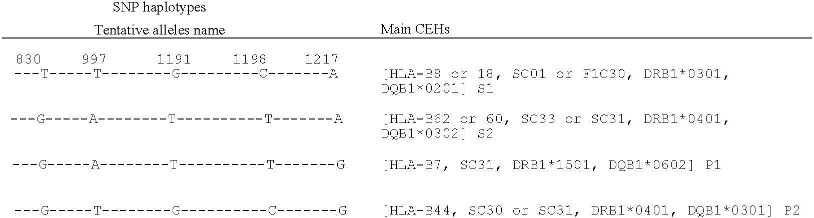

The 5-SNP haplotypes is used to study 17 instances of T1D patients who carry HLA-DQB1*062. The most common (n=8) SNP haplotypes in these patients appears to represent heterozygosity for S1S2 (see Table 3). There are 4 apparent homozygotes for S2 and 2 for S1. There are no instances of P1 in a T1D patient despite the presence of HLA-DQB1*0602 in all. Thus, all protective HLA-DQB1*0602 haplotypes in T1D patients studied show evidence of ancient crossovers centromeric to HLA-DQB1, most to a recognizable susceptibility SNP-defined allele.

TABLE 3
A first itron HLA-DOA2 5-SNP haplotypes on normal B7, DR@ and B8, DR3 CEHs
in T1D patients as well as controls and HLA-DOB1*0602-positive T1D patients

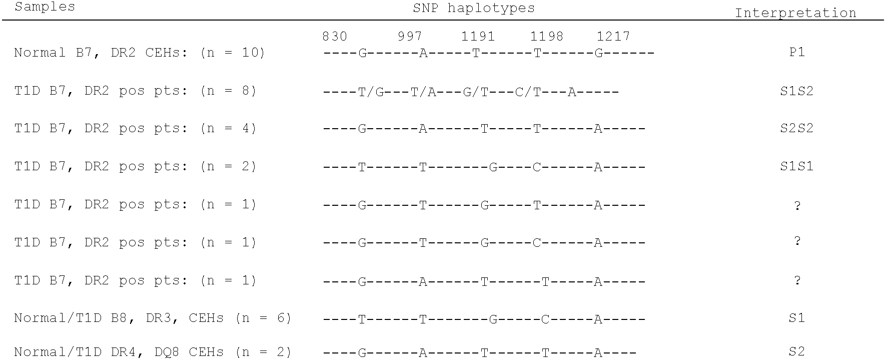

Explaining incomplete penetrance (i.e. why MZT are often discordant for T1D) might provide critical information toward understanding the genetics of this and other complex diseases. To understand penetrance, the mode of inheritance must be known. A method is developed and provides a hypothesis to explain incomplete penetrance of MHC disease susceptibility genes. Penetrance in MZT pairs is defined as intrinsic penetrance, characteristic of all completely genetically susceptible persons in the general population. Intrinsic penetrance is contrasted with apparent penetrance that varies with the number of shared susceptibility genes (apparent penetrance in MHC-identical sibs of patients>sibs in general>parents or children>random unrelated individuals). Given the fixity of DNA on CEHs, if a susceptibility allele occurs on a CEH within the region of fixity, as evidenced by its elevated frequency among patients compared with ethnically-matched controls, it occurs on virtually all independent examples of that CEH in the population.

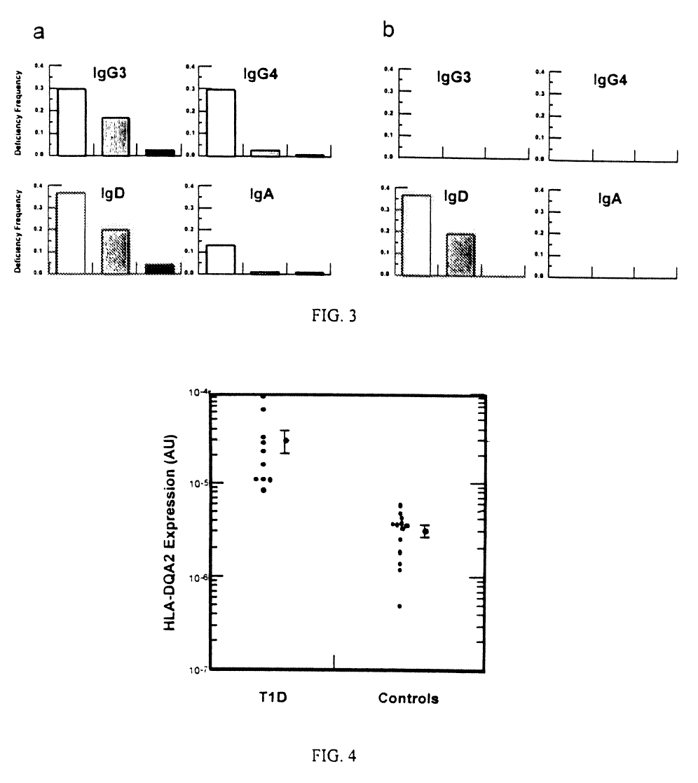

Based on this concept, the prospective method for analyzing the mode of inheritance and estimating apparent penetrance of genes on CEHs is developed. If homozygotes but few, if any, heterozgotes or non-carriers for a CEH exhibit an associated trait, the susceptibility gene carried by the CEH is expressed recessively. On the other hand, if both homozygotes and heterozgotes but few, if any, non-carriers express the trait, the susceptibility gene is dominantly expressed. Of homozygotes for the CEH [HLA-B8, SC01, DR3], approximately 13% had IgA deficiency (IgAd) and 30% had IgG4d, compared to 0.14% and 6.3% of controls, respectively (FIG. 3A). Heterozygotes and non-carriers of the CEH had rates similar to the general control population.

In contract, IgDd and IgG3d were common in both [HLA-B8, SC01, DR3] homozygotes (37% and 30% respectively) and heterozygotes (20% and 18% respectively) with the rates in homozygotes being nearly twice those of heterozygotes and much higher than controls (6.7% and 4.5% of the general Caucasian population, respectively). Thus, the results for IgDd and IgG3d suggested dominant MHC susceptibility genes for these traits. Because the IgDs occurred, in general, independently of each other and because similar Ig studies of another DR3-carrying CEH [HLA-B18, F1C30, DR3] revealed only IgDd (but not IgAd, IgG3d nor IgG4d (FIG. 3B)), it is clear that each of the Igd susceptibility genes is distinct from the others. Since the IgDd frequencies in [HLA-B18, F1C30, DR3] homozygotes (37%) and heterozygous (19%) are very similar to those in [HLA-B8, SC0, DR3] homozygotes and heterozygotes, (37% and 20% respectively), it may be that the susceptibility allele for IgDd is the same on both haplotypes.

Penetrance, if it were the result of an extrinsic triggering event, would be expected to affect the whole organism. For a dominant trait, it should be irrelevant for trait expression whether the host has 1 or 2 susceptibility alleles. On the other hand, if penetrance is an intrinsic property of the susceptibility gene on each chromosome, homozygotes with 2 susceptibility alleles should have a higher rate (up to twice) compared to heterozygotes with only 1. Since the rates of IgG3d and IgDd in [HLA-B8, SC01, DR3] and of IgDd in [HLA-B18, F1C30, DR3] homozygotes are almost twice those in heterozygotes, this suggested a stochastic basis for penetrance affecting homologous genes on each chromosome. In addition, it appears that penetrance is manifestation of the MHC susceptibility genes, since the only difference between the subjects is in their MHC haplotypes. If MHC genes are recessively expressed, penetrance is the same for the extrinsic trigger and intrinsic switch mechanisms, providing no information. However, it is likely that the intrinsic switch is also operative in recessive as well as dominant MHC trait expression.

Intrinsic penetrance for multiple MHC gene-determined traits in the same MZT subjects is examined by assaying IgA, IgD, IgG3 and IgG4 concentrations in serum from 50 pairs of MZT discordant for T1D. The frequencies of subjects deficient in IgA (6%), IgD (33%) and IgG4 (12%) are markedly to moderately higher in the MZT than in normal subjects, whereas IgG3d is comparable in frequency. This may be because the MHC haplotypes (as well as possible non-MHC genes and haplotypes) that predispose to T1D often also carry susceptibility genes for certain immunoglobulin deficiencies. Pairwise concordance for the deficiencies in the twins was 50% in IgAd, 57% in IgDd and 50% in IgG4d. Immunoglobulin deficiencies are not associated with the presence of diabetes. There are no significant deviations from expected random associations among the specific immunoglobulin deficiencies except that all IgAd subjects have IgDd. These results support the concept that intrinsic penetrance is a random process independently affecting different MHC susceptibility genes and alleles. While such observations do not rule out environmental triggers for penetrance, they make them far less likely, since one would require 5 different external environmental triggers (1 for each incompletely penetrant disorder) to explain the observations in MZT.

B cells are known to constitutively express HLA-DQA2, independently of the MHC class II transactivator. Using qRT-PCR, HLA-DQA2 transcription is approximately 10-fold higher in T1D patients' B cells compared with B cells from normal controls (FIG. 4). In 4 pairs of MZT (Table 4) concordant for T1D, HLA-DQA2 transcription is high, whereas in 3 pairs of discordant twins, the 3 T1D patients have high expression and the 3 healthy twins have low expression. There is no correlation between HLA-DQA1, transcription and the presence or absence of T1D. Of 7 normal homozygotes for the B8, DR3CEH and for the HLA-DQA2*S1 allele, 4 have normal HLA-DQA2 transcription but 3 have levels close to the median in T1D patients. Although these are very small sample numbers, finding 3 non-T1D control HLA-DQA2*S1 homozygotes out of 7 (43% observed vs. 30 to 50% predicted) with elevated HLA-DQA2 transcription in the range of that in T1D patients is crucial and consistent with the hypothesis. In studies of two subjects (1 hyperexpresser, 1 normal expresser), HLA-DQA2 expression by an equal number of B cells in PBMC and in B cell lines is the same.

TABLE 4
Transcription of HLA-DQA2 and HLA-DQA1 in MZT Concordant and Discordant for T1D

| a. Concordant for T1D | | | | b. Discordant for T1D | | | |
|---|---|---|---|---|---|---|---|
| DQA2 hi | DQA2 low | DQA1 hi | DQA1 low | DQA2 hi | DQA2 low | DQA1 hi | DQA1 low |
| Pair 1-1 | XX | | | | | | X |
| | | XX | | XX | | | |
| Pair 1-2 | XX | | X | | X | XX | |
| Pair 2-1 | XX | | X | | | | X |
| | | | | XX | | | |
| Pair 2-2 | XX | | X | | X | | X |
| Pair 3-1 | XX | | | | | | X |
| | | XX | | XX | | | |
| Pair 3-2 | XX | | X | | X | | X |
| Pair 4-1 | XX | | | | | | |
| | | XX | | | | | |
| Pair 4-2 | XX | | | | | | |
| | | XX | | | | | |

The picture of T1D MHC susceptibility that emerges is of a monomorphic protein encoded by a gene (H1A-DQA2) with an extraordinarily polymorphic first intron and 5′ UTR. Certain sequences appear to be subject to random epigenetic change that results in an order of magnitude increase in transcription in about 40% of susceptibility allele homozygous individuals, whether they are normal or have all the susceptibility genes needed for complete susceptibility of T1D. In the latter cases, T1D results, whether the T1D-susceptible individuals are unrelated or MZT of T1D patients. T1D patients all (the number so far studied in small) appear to be mostly homozygous or doubly heterozygous for 2 susceptibility alleles and no patient carries the main protective HLA-DQA2*P1 and P2 alleles carried by HLA-DQB1*0602 or DR4, DQB1*0301 haploytypes, respectively, in normals. This, combined with apparently universal high expression in patients provides strong preliminary evidence that HLA-DQA2 is the T1D MHC susceptibility locus.

This invention is further illustrated by the following examples which should not be construed as limiting the scope of the appended claims. The contents of all references, patents and published patent applications cited throughout this application, are incorporated herein by reference.

### EXPERIMENTAL

### Research Design and Methods

A number of hypotheses are tested concerning the identity of the MHC T1D susceptibility gene and its SNP-defined alleles, and the relationship between these alleles and expression levels of HLA-DQA2 and the occurrence of T1D.

### Hypotheses 1:

With regard to the identity of the MHC T1D susceptibility gene and its alleles, it is hypothesized that:
(a) There is a single MHC susceptibility locus for T1D.
(b) The mode of inheritance of T1D susceptibility alleles at this locus is Mendelian recessive
(c) The T1D susceptibility locus is centromeric to HLA-DQB1.
(d) The T1D susceptibility locus is HLA-DQA2.
(e) The fixity of strong susceptibility CEHs extends through the HLA-DQA2 susceptibility locus in patients and most controls.
(f) The fixity of protective CEHs usually includes the HLA-DQA2 T1D susceptibility locus in control but not in patients, in whom an ancient crossover has occurred centromeric to HLA-DQB1 such that the haplotypes now carries a susceptibility allele at HLA-DQA2.
(g) Neutral DR haplotypes in both control individuals and T1D patients are the result of lack of fixity centromeric to HLA-DRB1, -DQB1.
(h) All patients are homozygous for HLA-DQA2 susceptibility alleles. No patient carries an HLA-DQA2 protective allele.
(i) There may be several different alleles at the susceptibility locus: at least 1 susceptibility allele (S1) for [HLA-B8, SC01, DR3] and [HLA-B18, F1C30, DR3] and 1 susceptibility allele (S2) for (HLA-DRB1*04, DQB1*0302-containing CEHs) as well as 1 protective allele (P1) for [HLA-DRB1*1501, DQB1*0602] and 1 (P2) for (HLA-DRB1*04, DQB1*0301). It is theoretically irrelevant whether these represent 4 or more distinct alleles or whether there is only 1 allele for susceptibility and 1 allele for protection. Each of these distinct susceptibility and protective alleles may represent sets of alleles with additional variation at non-differentiating sites.
(j) Among random normal European Caucasian controls, the aggregate frequency of susceptibility-conferring HLA-DQA2 alleles at the susceptibility locus is 0.525.
(k) For polymorphic loci centromeric to the susceptibility locus itself, some T1D patients have protective marker alleles. Whether the frequencies are the same in patients and controls depends upon the extent of DNA fixity between HLA-DQA2 and the centromeric loci. If fixity is 100% the centromeric locus susceptibility and protective allele frequencies would be indistinguishable from the previously determined susceptibility locus and protective allele frequencies. If fixity is less than 100%, this confirms that the previously determined susceptibility locus is the true susceptibility locus.
(l) From affected sib pair analysis, in 55% of families, both parents are mostly HLA-DQA2*S heterozygotes, in 38% of families, usually 1 parent is homozygous, the other heterozygous. In 7% of families, both parents are HLA-DQA2*S allele homozygotes.

Hypotheses 2:

With regard to the relationship of the MHC T1D HLA-DQA2 susceptibility gene and gene expression, it is hypothesized that:
(a) Elevated transcription of HLA-DQA2 is a stochastic epigenetic event.
(b) Elevated or low transcription of HLA-DQA2 is related to HLA-DQA2*S vs. *P alleles. Only homozygotes for HLA-DQA2 susceptibility-conferring (S) alleles are subject to increased expression of HLA-DQA2.
(c) HLA-DQA2 expression rates may differ in homozygotes for different susceptibility-conferring alleles HLA-DQA2*S1 and HLA-DQA2*S2 (the observed overall rates may be averages).
(d) Increased expression of HLA-DQA2 occurs in all T1D patients.
(e) Of normal homozygotes for HLA-DQA2 susceptibility alleles, 40% (the same as MZT of a patient) have elevated expression of the HLA-DQA2 gene. Such normal persons do not have T1D because they presumably lack the other susceptibility genes necessary to have T1D. Normal subjects with high HLA-DQA2 expression probably do not have symptoms.
(f) Given that the aggregate frequency of HLA-DQA2 susceptibility alleles in the general population is calculated as 0.525, homozygotes for susceptibility have a frequency of 0.276 (0.525²) in the general population.
(g) If 11% (0.276×0.40=0.11) of normal individuals have high HLA-DQA2 expression rates, the elevated expression leads to T1D in fully susceptible persons and is not secondary to T1D.
(h) Elevated transcription of HLA-DQA2 is always accompanied by increased translation.
(i) Changes in chemical modification (methylation/demethylation, for example) or other regulatory effects of regions in the first intron of the HLA-DQA2 gene result in the observed 10-fold increase in HLA-DQA2 expression in T1D patients.
(j) The increased HLA-DQA2 protein may exist as a homodimer or heterodimer with another class II beta chain, including the normally non-expressed HLA-DQB2 or HLA-DQB3.

### Cell Separation and Flow Cytometry:

Mouse monoclonal antibodies and immunomagnetic beads coated with goat anti-mouse Ig (Miltenyi Biotech, Heidelberg, Germany) or fluorescence-activated cell sorting with appropriate mouse monoclonal antibodies are used to provide populations of cells highly enriched in the desired cells. The separated cells are assayed for purity by flow cytometry using antibodies characteristic of potential contaminating cell lineages.

### CD19+B Cell Purification:

PBMC are obtained from venous blood samples of T1D and normal donors after Ficoll-Paque (Pharmacia, Piscataway, N.J.) gradient centrifugation. Cells are either used immediately or frozen in a solution of 10% heat-inactivated pooled human serum (PHS) (Gemini, W. Sacramento, Calif.), 10% dimethylsulfoxide (DMSO), 1% penicillin-streptomycin and 1% L-glutamine (all from Cellgro, Fisherbiotech) in RPMI. PBMC are thawed (if frozen) and washed twice with a medium of 44% DEMEM (BioWhittaker, Walkersville, Md.), 44% F12 (Life Technologies, Rockville, Md.), 10% heat-inactivated fetal calf serum (Atlanta Biologicals, Norcross, Ga.), 1% pen/strep, and 1% L-glutamine. Before CD19+ cell selection, non-specific binding is minimized by resuspending cells in a solution of 10% human IgG (CBR Laboratories, Inc., Boston, Mass.), 10% heat-inactivated PHS in AIM V serum-free media at 100 ul per 10×10⁶ cells and incubating on ice with rocking for 30 min. After blocking for non-specific binding, cells are washed with PBS, and MACS anti-CD19 beads (Miltenyi Biotec Inc., Auburn, Calif.) are added at 20 ul beads per 10×10⁶ cells. Cells are incubated on ice for 30 min and sorted on a Miltenyi Automacs instrument. The CD 19+ fraction is collected and counted with trypan blue.

Yield is generally 5-15% of PBMC.

### Preparation of B Cell Lines:

Large quantities of virus are produced by EBV-infected marmoset B cells. Marmoset cell line B95-8 (originally obtained from ATCC, Rockville, Md.) is used. B-LCL (B lymphoblastoid cell lines) are established by deliberate infection of peripheral blood B cells with exogenous EBV harvested from cultures of the B95-8 marmoset cell line. Fresh or frozen PBMCs can be used. PBMCs are suspended in 10% FCS in RPMI with 1% penicillin/streptomycin and 1% L-glutamine, to a concentration of 4×10⁶ mL. An equal volume of EBV supernatant is added to the resuspended cells. One mL of cell suspension is placed in each well of a 24-well plate, using every other row to prevent cross-contamination. Two mg of cyclosporine A is added per mL of cell suspension. The addition of cyclosporine inactivates T cells. Thus, this helps to eliminate any regression of growth caused by cytotoxic T memory cells that recognize virus-encoded cell surface antigens. This mixture is incubated at 37° C. with 95% humidity and 5% CO₂ for 1 wk without disturbance. On the seventh or eighth day 0.5 mL of 10% FCS in RPMI medium is added. The culture is then incubated for another 3 days and then 0.5 mL of media is removed from each well and replaced with 0.5 mL of media is removed from each well and replaced with 0.5 mL 10% FCS in RPMI. This is repeated until 0.5-1.0 mm (visible) colonies form.

### Nucleotide Sequencing:

PCR products obtained from RT-PCR and uncloned or cloned genomic DNA is sequenced for verification of nucleotide sequences. Sequencing is performed directly on PCR products or following cloning into TA cloning vector (Invitrogen, Carlsbad, Calif.). For cloned products, DNA is isolated from minipreps using Qiagen kits (Qiagen, Valencia, Calif.) and sequencing is carried out at Davis Sequencing (Davis, Calif.) by the Big Dye Terminator method using an ABI/Perkin-Elmer 3730 automated sequencer.

Quantitative Real-Time PCR to Determine Transcription Rates of HLA-DQA2 in B Cells from T1D Patients and Controls:

The expression of HLA-DQA2 in B cells and B cell lines is examined (determining first that rates are the same in fresh B cells and B cell lines from the same individual and stable over time). RNA is isolated from EBV B cell lines (or CD19+B cells) and cDNA is synthesized using M-MLV reverse transcriptase. Quantitative RT-PCR is used to determine the rates of transcription of HLA-DQA2 in B cells from T1D patients and age and sex-matched controls. Quantitative RT-PCR is performed with SYBR Ex Taq HS from Takara Mirus Bio (Madison, Wis.). SYBR Green I fluorescent dye is a sequence-independent, universal qRT-PCR detection reagent with the ability to bind to all dsDNA molecules. Real-time PCR primers are designed to assure maximal efficiency and sensitivity based on primer-3 and Oligonucleotide Properties Calculator programs. Real-time PCR is performed in the iCycler iQ PCR detection system (Bio-Rad, Hercules, Calif.) using the following program: 10 sec of pre-incubation at 95° C. followed by 40 cycles for 5 sec at 95° C. and 30 sec at 60° C. Individual real-time PCR reactions are carried out in 20 μL volumes in a 96-well plate containing 1×SYBR Premix Ex Taq, 0.2 μM forward primer, 0.2 μM reverse primer and varying amounts of template cDNA as described by the manufacturer (Takara). GAPDH expression provides the internal standard. Amplification of the highly homologous HLA-DQA is avoided. Specific forward and reverse primers for unique HLA-DQA2 and HLA-DQA1 consensus exons (avoiding allelic differences) for these experiments are known. Controls include RT-PCR of HLA-DRA1, HLA-DRB1, HLA-DQA1 and HLA-DQB1 in the same samples, we well.

mRNA Extraction and RT-PCR for HLA-DRA1, HLA-DRB1, HLA-DQA1 and HLA-DQB1 Genes.

RNA is isolated using the Ultraspec RNA reagent (Biotecx Laboratories, Houston, Tex.) and cDNA synthesized with M-MLV reverse transcriptase. PCR is performed using Taq polymerase (2.5 U), MgCl₂ (1.5 mM), and dNTP (0.4 mM) with cycling conditions described as follows for GAPDH and the MHC class II genes: 94° C. for 30 sec, 58° C. for 30 sec, 72° C. for 1 min with a final extension at 72° C. for 7 min. The following conditions are used: 2 cyles of 96° C. for 1 min and 58° C. for 4 min; 30 cycles of 94° C. for 1 min and 58° C. for 2.5 min, followed by extension at 70° C. for 10 min and soak at 25° C. The following primer sets are used: GAPDH, 5′-CGACCACTTTGTCAAGCTCA-3′ (SEQ ID NO: 1) and 5′-AGGGGTCTACATGGCAACTG-3′ (SEQ ID NO: 2) HLA-DQA1,5′-GGTGTAAACTTGTACCAGT-3′ (SEQ ID NO: 3) and 5′-CCTTGGTGTCTGGAAGCACCAACTGA-3 (SEQ ID NO: 4); HLA-DRB1,5′-CTCCAGCATGGTGTGTGTCTG-3′ (SEQ ID NO: 5) and 5′-GCTGGGTCTTTGAAGGAT-3′ (SEQ. ID NO: 6); HLA-DQB1, 5′CGCTTCGACAGCGACGT-3′ (SEQ ID NO: 7) and 5′CCAC-CTCGTAGTTGTGTCTGCA-3′ (SEQ ID NO: 8); HLA-DRA, 5′GCCAACCTGGAAATCATGACA-3 (SEQ ID NO: 9). PCR products are visualized under UV in 2% ethidium bromide-stained agarose gels. Primers for HLA-DQB2 and HLA-DQB3 expression are designed based on exon consensus sequences.

### Methylation Specific PCR:

DNA is modified by sodium bisulfate treatment converting unmethylated, but not methylated, cytosines to uracil. Following removal of bisulfate and completion of the chemical conversion, this modified DNA is used as a template for PCR. Two PCR reactions are performed for each DNA sample, one specific for DNA originally methylated for the gene of interest, and one for unmethylated DNA. PCR products are separated on 6-8% non-denaturing polyacrylamide gels and the bands are visualized by staining with ethidium bromide. The presence of a band of the appropriate molecular weight indicates the presense of unmethylated, and/or methylated alleles, in the original sample. For each 50 μL reaction, the following amounts are used: 5 μL of 10×PCR buffer; 2.5 μL of 25 mM dNTP mix; 1 μL of antisense primer (300 ng/μL); 28.5 μL of dd water. 38 μL of this PCR mix are aliquoted into separate PCR tubes. 2 μL of bisulfite modified DNA template is added to each tube. One to two drops of mineral oil are added to each tube, and placed in the thermocycler. The PCR is initiated with a 5 min denaturation at 95° C. Taq polymerase is added (1.25 units in 10 uL of dd water) to 40 L of mix. The 10 μL is mixed into the 40 μL though the oil by gently pipetting up and down. Other methods of hot-start PCR may be more convenient. Amplification is continued with the following parameters (35 cycles): 30 sec at 95° C.; 30 sec for specific primer annealing; 30 sec at 72° C. for elongation. A final extension is carried out for 4 min at 72° C. PCR products are analyzed by electrophoresis on 6-8% non-denaturing polyacrylamide gels. DNA typing for HLA class I (A, Cw and B) and class II (DRB3/4/5, DRB1, DQA1, DQB!, DPB1) alleles by chemiluminescent sequence-specific oligonucleotide probe hybridization (SSOPH):

For immediate to high resolution HLA typing of multiple samples, a chemiluminescent detection method is used. This method employs a large series of SSOs for each locus, conjugated with alkaline phosphatase (AP) to identify HLA class I and class II alleles amplified by PCR, as described above. Genomic DNA is extracted from fresh or frozen whole blood, PBMCs or from B-LCL using a QIAamp mini-prep kit (Qiagen, Valencia, Calif.). The primers used are specific for each HLA locus to be typed. These are based on the 12^{th} International Workshop and are updated with new alleles via the NCBI website. The DNA Thermal Cycler (Perkin Elmer 9600/9700) is programmed appropriately for each target. Optimal denaturation, annealing and extension temperatures have been established. The optimum concentration for MgCl₂ has also been determined. All samples are checked for amplification of DNA by electrophoresis in a 3% agarose gel containing ethidium bromide (0.5 mg/mL). About 40-100 ng of DNA is obtained from each amplification. About 50 ng of amplified DNA is spotted on a positively charged nylon membrane. After drying at room temperature, the membranes are wet with 0.4 N NaOH for 5 min to denature. After an additional 10 min in 10×SSPE, the membrane is dried and then illuminated with a 254 nm-UV lamp to 0.12 Jcm². The membranes are pre-wet in 20 mL of 2×SSC for 5 min at room temperature. Quick-Light™ Hybridization Solution (Tepnel Lifecodes, Stamford, Conn.) pre-heated to 45° C., is combined with 25 uL of probe solution. The 2×SSC solution is discarded and the prehybridization solution containing the labeled probe is added. The mixture is incubated in a shaking water bath for 30 min at 45° C.

The probe-hybridization solution is discarded and 100 mL of 2×SSC are added and rotated for a few min at room temperature. TMAC wash solution (50 mM Tris-HCl, 3.0M tetramethylammonium chloride (TMAC)) containing 0.1% SDS is added (preheated to 60° C.) and the membranes are rotated in a shaking water bath for 20 min. Finally, 100 mL of 2×SSC is used to rinse the membranes. The membranes are placed in a solution of 1× Quick-Light™ for 1 min. This is repeated 3 times with new Quick-light solution each time. The membrane is then placed in a development folder and sprayed with Lumiphos®. The folder is then exposed to X-Ray film for 0.5-2 h. The resulting films are scanned and the results are recorded and stored on computer. The analysis and interpretation of results is done with special software and the patterns that are obtained are compared with a current database for all HLA alleles. PCR for first intron SNP haplotypes determination:

Four sets of primers are currently used for the 5-SNP haplotype given in Tables 2 and 3. These are:

| | |
|---|---|
| 8.2F: | |
| 5′-TCAAAGACTAGGGATGTTTCCA-3 | (SEQ ID NO: 10) |
| 8.2R: | |
| 5′-CATAAAGCATATCTACATGCCCATTT-3 | (SEQ ID NO: 11) |
| 8.3F: | |
| 5′-AAATGGGCATGTAGATATGCTTT-3 | (SEQ ID NO: 12) |
| 8.3R: | |
| 5′TTCCAGTTCACTTCCCCTTTT-3 | (SEQ ID NO: 13) |
| 8.3-1R: | |
| 5′TCAGAATGGGGAGCAGCTATG-3 | (SEQ ID NO: 14) |
| 8.4F: | |
| 5′-TAACCATAGCTGCTCCCATTC-3 | (SEQ ID NO: 15) |
| 8.4R: | |
| 5′-AGCTACTCGGGGAGGCTGAG-3 | (SEQ ID NO: 16) |

### C4, C2, and BF Typing:

The intermediate region of the MHC contains 4 genes for the complement proteins C2, factor B, C4A and C4B. These 4 genes occur as units called complotypes. No well-documented crossover has been reported within the complotype, and they are important definers of extended haplotypes since most of the latter have unique comploytpes.

### Statistics:

Comparisons of subjects in various categories such as observed and expected frequencies are analyzed for significance by X² test or by Fisher's exact test if cells <6 are involved. For testing for differences in mean expression levels, no statistical test is needed since there is no or minimal overlap between high and low levels and the means differ by an order of magnitude. Other quantitative data are compared and p values calculated by Student's test. For the testing of the prediction that the aggregate normal frequency of all HLA-DQA2 susceptibility alleles is 0.525, the ability to detect a proportion of 0.455 or lower with a power of 80% at alpha=0.05 requires 399 family control haplotypes (200 families or 200 random normal subjects).

Because the fixity of DNA on intact susceptibility marker CEHs confounds attempts to localize susceptibility genes, they are used in patients only to (a) detect the extent of fixity centromeric to HLA-DQB1 and (b) define susceptibility alleles at positional candidate loci. Protective CEHs in normals are used in the same way to define protective alleles. For localization, alleles of positional candidate susceptibility genes on protective HLA-DQB1 haplotypes occurring in T1D patients are studied. The study of these can reveal ancient (prior or ancestral) crossover to susceptibility alleles instead of the protective alleles in normal subjects. Almost as informative are non-DR2, non-DR3, non-DR4 and non-DR7 haploytpes since it is hypothesized that they carry only susceptibility alleles of the true susceptibility locus in patients but a mixture of susceptibility and protective alleles in normal subjects.

### Example 1

### Purpose

To determine whether ancient crossovers centromeric to HLA-DQB1 involve HLA-DQB3, HLA-DQA2 and/or HLA-DQB2 in protective and neutral CEHs in T1D patients.

### Rationale:

To show that a SNP haplotypes present in a candidate gene in almost all instances of a protective CEH in normal subjects is not present in the T1D patients who carry it, i.e., there has been an ancient crossover between HLA-DQB1 and the candidate locus. For neutral HLA-DR, -DQ haplotypes, show that there is little DNA fixity centromeric to HLA-DQB1 in either patients or controls.

### Experimental Design:

Using the Sanger Centre sequences as well as sequences for HLA-DRB1*04, -DQB1*0302-containing CEHs, SNPs are selected for analysis that distinguish genes on the protective haplotypes. For example, on HLA-B7, DR2 vs. B8, DR3, there are at least 42 such SNPs for HLA-DQB2 and there are 29 for HLA-DQB3. The consensus SNPs at each locus for each strongly protective CEH is determined in at least 25 independent examples in normal controls. There is access to 23 DNA samples from T1D patients who carry HLA-DQB1*0602. These samples are from studies of T1D patients and samples from collaborators who reported on the normal nucleotide sequences in 14 such patients. It is expected that at least as many total HLA-DRB1*04, DQB1*0301; HLA-DRB1*07, -DQB1*0202; and HLA-DRB1*07, -DQB1*0303 haplotypes in T1D patients from the same sources and normal MHC-typed controls are available. 25 patients who carry the protective HLA-DQB1*0602 for SNP haplotypes are studied. Any differences in consensus SNPs on normal haplotypes and comparable SNPs in T1D patients are considered to represent crossovers in the patients. When information on the 3 loci to be examined is available, this attribution can be further checked. In other words, if a postulated crossover occurs involving HLA-DQB3, it is to be expected that departure from consensus SNPs will also occur at the centromeric loci HLA-DQA2 and HLA-DQB2. Since it is suspected that HLA-DQA2 is the susceptibility locus, the informative SNPs described in C5 is used to identify ancient crossovers in T1D patients to minimize effort and maximize information.

It is hypothesized that the reason that HLA-DR1, DR5 and DR6 haplotypes are neutral or weak markers for T1D is that they show relatively little fixity between HLA-DRB1, -DQB1 and the susceptibility locus. This hypothesis is tested in a total of 25 each of HLA-DR1, DR5 and DR6 (neutral and mildly protective) haplotypes in normal subjects and in 25 T1D patients. For these, the same SNPs in HLA-DQB3, -DQA2 and -DQB2 are analyzed as for the protective haplotypes with the aim of showing diversity of SNP haplotypes in all 3 loci (lack of fixity) in patients and in controls.

### Results:

The outlined experiments reveal where the critical ancient crossovers are that result in substitutions of protective alleles by susceptibility alleles at the true susceptibility locus. It is expected to be able to show definitely that, for all protective HLA-DR, -DQ haplotypes in patients, ancient crossovers from SNP haplotypes-defined protective alleles have occurred. If fewer than 100% of crossovers occurred telomeric to or at HLA-DQB2, the analyses are extended to centromeric genes until 100% is reached. (A further test of this is part of Example 2 in which any protective allele of the true susceptibility locus occurs in any T1D patient). An important expected result of these studies is the determination of the extent of fixity of alleles over the 3-gene region and between it and HLA-DQB1. It is possible that some DR1, DR5 and DR6 haploypes are protective and others are susceptibility-conferring, despite having the same HLA-DQB1 alleles, and neutrality results from these effects balancing each other. This possibility is tested by analyzing [B35, SC30, DR1]; [B65, SC2(1,2), DR1]; [B35, SC31, DR5]; [B18, SC31, DR5] and [B60, SC02, DR6] CEHs in controls and patients for SNP diversity separately.

### Example 2

### Purpose

To determine whether the true susceptibility locus is HLA-DQA2, and show that (a) all T1D patients are homozygous for HLA-DQA2 susceptibility alleles and none has a protective allele of HLA-DQA2 and (b) flanking polymorphic genes (HLA-DQB3 and HLA-DQB2) do not satisfy these stringent criteria.

### Rationale:

The overall approach is to identify SNP haplotypes that distinguish susceptibility (S) from protective (P) alleles (informative SNPs) at the HLA-DQA2 locus. To do this, consensus sequences in the HLA-DQA2 first intron (and 5′UT, if needed) of susceptibility and protective CEHs in normal control subjects and in T1D patients is determined. The marker CEH is largely fixed through HLA-DQA2 and possibly HLA DQB2, and should thus provide the major S and P alles for each locus. The SNP data from Example 1 for normally protective CEHs is used to define HLA-DQA2*P1 to *P4. In randomly selected T1D patients, marker CEHs (−65% of the total haplotypes) invariably or nearly invariably have the S alleles of the marker CEHs at the susceptibility locus and define HLA-DQA2*S1 and *S2. It is the remaining 35% or so of haplotypes in T1D patients provide the key information regarding the invariability of S alleles. The neutral (DRX, DQY) and protective haplotypes in patients are the best potential sources of non-HLA-DQA2*S1 or *S2 susceptibility alleles.

### Experimental Design:

The first task is to define specific susceptibility-conferring and protective SNP haplotypes for HLA-DQB3, -DQB3, -DQA2, and -DQB2. Tentative information main susceptibility-conferring HLA-DQA2 SNP haplotypes (S1 on B8 or B18, DR3 and S2 on DR4, DQ8 CEHs, and protective P1 or DR2, DQ6 and P2 on DR4, DQ7 CEHs, see Table 2) is established. Given the fixity on CEHs and fragments of CEHs, the burden of proof for T1D patients being largely HLA-DQA2*S1S1, *S1S2 or *S2S2 and never having a P allele must reside in DRX, DQY carriers (the HLA-DR, DQ non-DR2, -3, -4, -7, i.e., non-marker haplotypes). Especially informative are those patients with the usually protective DR2, DQB1*0602 or DR4, DQB1*0301 and other haplotypes identified and studied in Example 1. The data from all of these studies and additional SNPs, as necessary, are used to define HLA-DQA2*S alleles. Informative SNPs on the protective DR7CEHs [HLA-B44, FC31, DR71, DQB1*0202] and [HLA-B57, SC61, DQB1*0303] on healthy control haplotypes is determined to define possible additional HLA-DQA2*P alleles (*P3 and *P4).

For the PCRs, it is necessary to have a sequence cloned DNA in the bulk of samples since most patients (about 88%) have at least 1 CEH with a defined and constant sequence. Moreover, because families are studied, phase assignment of SNP haplotypes in relation to other MHC markers is usually definitive. Many subjects have been MHC(HLA-A, -B, complotype, -DRB1, -DQB1) typed. MHC is typed for those who have not.

Informative SNP haplotypes on a sufficient number of independent examples of DRX, DQY in controls or patients or both, as appropriate (at least 25 examples of each haplotypes), is determined to assure that the informative SNPs mark all or nearly all instances of the HLA-DQA2*S or *P alleles. SNP haplotype-defined S and P alleles in the total randomly selected 200 T1D patients and their relatives is determined to produce over 400 T1D and about 400 control haplotypes. Faced with the formidable task of selecting informative SNPs from the 49 that distinguish the HLA-DQA2 first intron on the 4 haplotypes studied by the Sanger Centre, those SNPs that distinguish HLA-DQA2 on B8 or B18, DR3 and DR4, DQ8 CEHs from those on B7, DR and DR4, DQ7 CEHs (informative SNP haplotypes, see Table 2) are studied. Because the Sanger Centre analysis did not include nucleotide sequences on an HLA-DRB1*04, DQB1*0302 haplotype, the nucleotide sequences of the first intron of HLA-DQA2 in a presumed HLA-DQA2 homozygous T1D patient who had 2 CEHs with HLA-DRB1*0401, HLA-DQB1*0302 is determined. In keeping with the CEH concept, nucleotide sequences on these 2 independent haplotypes are identical over the entire 3.4 kb first intron. For the DR4, DQ7 nucleotide sequence, it is assumed that the Sanger Centre sequence applies to the bulk of DR4, DQ7 HLA-DQA2 first intron sequences in normal subjects. As informative SNPs are determined in independent instances of DR4, DQ7 haplotypes in normal subjects, these consensus sequences will be modified or kept. Two PCR amplicons are subjected to nucleotide sequence analysis that distinguish HLA-DQA2*S1, *S2, *P1 and *P2 from each other. These PCRs are extended as needed.

If no HLA-DQA2*P allele among 400 T1D patient haplotypes are found, HLA-DQA2 is provisionally considered to be the MHC T1D susceptibility locus. It is suggested that HLA-DQA2*S1 and *S2 (see Table 2) are the major susceptibility alleles of HLA-DQA2 in Caucasians. If these are the only SNP haplotypes found among DNA samples from a sufficient (initially 200) number of randomly selected T1D patients and no T1D patient carries HLA-DQA2*P1, *P2, *P3 or *P4, this presumption is considered true and simple Mendelain inheritance for these susceptibility alleles is established. The HLA-DQA2*S1 and *S2 alleles may represent sets of alleles with nucleotide variation at points other than the SNP haplotypes used to define them. In this case, it is necessary to show that the functional classification (and eventually, chemical or other modification) corresponds to the distinguishing (informative) SNP haplotypes.

To establish this unequivocally, HLA-DQB3 and HLA-DQB2 informative polymorphisms that distinguish marker susceptibility CEHs are analyzed to define S and P alleles for those loci (determined as detailed above for HLA-DQA2). These are tested in the T1D patients with protective DR, DQ haplotypes studied in Example 1. Even if all T1D patients are homozygous for susceptibility alleles of HLA-DQA2, it must still be shown that the flanking genes, HLA-DQB3 and HLA-DQB2 (expressed or unexpressed), are not invariably represented by S alleles in T1D patients. This is particularly the case on non-CEH-related MHC haplotypes (HLA-DRX, -DQY and protective HLA-DR, -DQ in patients), but studies in a relatively small number (more than 100) of such haplotypes should suffice to make the point. In essence, if the frequency of P alleles for HLA-DQA2 among T1D haplotypes is 0 or significantly less than P alleles for either HLA-DQB3 or HLA-DQB2, HLA-DQA2 is the MHC T1D susceptibility locus.

The classification of P alleles is more complicated than S alleles because they are all the HLA-DQA2 non-S alleles. From a practical point of view, it should be enough to demonstrate that the CEH-defined P alleles (HLA-DQA2*P1-*P4) do not occur among randomly selected T1D patients and/or that all HLA-DQA2 alleles at the informative loci are S alleles to prove the point. For completeness, the full first itron and 5′UT HLA-DQA2 sequences for S1, S2 and the high frequency P alleles is determined.

The prediction that the frequency of HLA-DQA2*S alleles in the overall normal population is approximately 0.525 is tested. From family studies of T1D patients, approximately 400 family (automatically matched for ethnicity) control haplotypes with defined HLA-DQA2 informative S and P alleles is reviewed. At a power of 80% and alpha=0.05, this number of samples (or 200 random normal subjects) gives the ability to prove the point with an observed frequency as low as 0.455. At an overall frequency of 0.525 for aggregate S allele frequency, HLA-DQA2*S homozygote frequency should be 0.275. There is evidence in at least 45% of families for parental homozygosity for T1D MHC susceptibility alleles. This is tested in multiplex families, including those with various degrees of MHC haplotype sharing by the affected sib pairs. First-degree relatives of up to 30 families with 2 affected sibs are studied. In 10 of these families, the sibs are MHC identical, in 10 they are haploidentical and, in up to 10, they share no MHC haplotypes with the patient. The object of these studies is to show that, for the first set, the parents are largely heterozygous for HLA-DQA2*S alleles, in the second set, one parent is usually heterozygous and, in the third, both are homozygous for HLA-DQA2*S alleles.

### Results:

The biggest problem is to identify the critical SNPs that define the HLA-DQA2*S and *P alleles. In the end, the final identification might only come after the accomplishment of Example 3 and the determination of which nucleotides are chemically altered or otherwise act to change the level of HLA-DQA2 expression. Given the extreme polymorphism of the HLA-DQA2 first intron, the bases for this variation should be understood. The demonstration that only certain HLA-DQA2 alleles are subject to upregulation in patients with T1D and in normal subjects gives insight into the nature and mechanism of penetrance, but also helps decide which polymorphisms are of little functional importance.

If a non-HLA-DQA2*S1 or *S2 allele is found in a T1D patient, it is necessary to determine if it is a new P or S allele. If its frequency is lower or 0 in control haplotypes, it is considered a rare S allele. If it has a higher frequency among normal haplotypes, it should be determined whether this is also true for other HLA-DQA2 first intron or 5′UTR SNP haplotypes on this same HLA-DR, -DQ haplotype. If it is determined that any protective CEH or DR, DQ haplotypes in a T1D patient has its usual normal HLA-DQA2*P allele, this is evidence that the true MHC susceptibility is centromeric to HLA-DQA2. If HLA-DQB3, -DQA2 and -DQB2 form a fixed unit, but if HLA-DQB2 and -DQB3 are not expressed (Example 3) in patients' B cells (or any other tissue) it is likely that HLA-DQA2, as the expressed gene, is the susceptibility locus. If the 2 or 3-gene unit is fixed (i.e., no hotspot of recombination among them) but either HLA-DQB2 or -DQB3 is expressed in T1D patients, there is a problem determining which is the real susceptibility gene.

Another possible reason for an HLA-DQA2*P allele in a patient is unusual hyperexpression by a usually protective allele. If HLA-DQA2*S alleles have a 40% risk of hyperexpression, it is possible that the risk for an HLA-DQA2*P allele is not 0, but some very low percentage. It is important to carefully determine HLA-DQA2 expression in such a patient.

### Example 3

### Purpose

To determine the expression of HLA-DQA2 in T1D patients, controls, and T1D-condordant and discordant MZT in relation to SNP-defined HLA-DQA2*S and *P alleles.

### Rationale:

Penetrance of susceptibility genes in a T1D genetically susceptible HLA-DQA2*S homozygous person is associated with markedly increased expression of HLA-DQA2. Quantitative HLA-DQA2 transcription is studied in randomly selected T1D patients and controls typed for susceptibility-conferring and protective alleles of HLA-DQA2 gene (or other candidate susceptibility gene). It is desired to show this definitely in a larger group of MZT concordant and discordant for T1D. Finally, the questions related to HLA-DQA2 translation and to HLA-DQA2 protein in patients and normal subjects is explored.

### Experimental Design:

1. HLA-DQA2 transcription. The expression of HLA-DQA2 in B cells and B cell lines is examined to confirm that: (a) B cells are the predominant or exclusive blood cell expressing HLA-DQA2, (b) expression rates are stable in samples taken over time; and (c) expression rates are essentially the same, corrected for B cell number, in PBMC from the same individual in 5 separate experiments involving different donors. Choose one of these cellular sources for further studies based on quantitative RT-PCR determinations of transcription of HLA-DQA2. GAPDH expression will provide the internal standard. We already have specific primers for unique HLA-DQA2 exon sequences (that do not amplify the highly homologous HLA-DQA1 exons). We will perform the following experiments:

a. By correlating high HLA-DQA2 expression with the presence of homozygosity for HLA-DQA2*S alleles in 75 T1D patients and 75 normal controls (25 HLA-DQA2*S1S1, 25*S1S2 and 25 *S2S2), it is expected to show that all T1D patients and a fraction (about 40%) of the controls have high expression. By showing that normal homozygotes for HLA-DQA2*S alleles are subject to the same stochastic upregulation of HLA-DQA2 as T1D patients, it is demonstrated that higher HLA-DQA2 expression is not secondary to the presence of T1D but is it likely sine qua non for T1D to develop in genetically susceptible individuals. The mean level in the three groups of HLA-DQA2*S high expressors are compared for statistical significance. If the number of high expressers among the HLA-DQA2*S1S1 controls is higher than among the HLA-DQA2*S2S2 controls (or vice versa), with heterozygotes having an intermediate value, this suggests different stochastic rates of hyperexpression for the two alleles. If one allele is 60% and the other 20% then 23 samples are required at alpha=0.05 and 80% power to show this. If additional HLA-DQA2*S alleles are found in homozygous HLA-DQA2*S normal subjects in Example 2 or in the study of protective HLA-DR, -DQ or of DR1⁺, DR6⁺ patients, they are studied for their stochastic rate of hyperexpression.

b. To determine whether normal subjects who carry HLA-DQA2*P alleles are ever hyperexpressers, 50 normal subjects (25 HLA-DQA2*SP, 25*PP) are tested for HLA-DQA2 expression. It is not expected that any HLA-DQA2*P-positive subject show hyperexpression. In the unlikely event that a small percentage of these normal subjects have hyperexpression, whether this is true of HLA-DQA2*S1P, *S2P, or some other *SP or *PP controls is determined.

c. To determine if the prediction is approximately correct that 11% of normal subjects have a high expression of HLA-DQA2 in an analysis of 100 randomly selected control subjects.

d. B cells from 25 T1D patients, 25 healthy hyperexpressers and 25 healthy normal expressers of HLA-DQA2 are studied for expression of HLA-DQB3 and HLA-DQB2 to determine if either of these genes is expressed in T1D patients or normal subjects.

e. To determine HLA-DQA2 expression in 20 monozygotic twins concordant and 30 discordant for T1D. This further establishes that hyperexpression occurs in all twins with T1D. The experiment also tests whether non-MHC genes contribute to incompleted penetrance. This is one explanation for any healthy twin with HLA-DQA2 hyperexpression. Any such subject is tested for other predictors of impending T1D and followed over time to rule this possibility out. As controls for hyperexpression of HLA-DQA2 among twins, expression of other MHC class II genes is tested by qRT-PCT of HLA-DRA1, HLA-DRB1, HLA-DQA1 and HLA-DQB1.

2. B cell surface HLA-DQA2 protein. With no commercially available antibody to HLA-DQA2 protein, arrange for custom-made mouse hybridomas secreting monoclonal antibodies to recombinant HLA-DQA2-specific peptides. Antibodies are selected that react with HLA-DQA2 but do not react with recombinant normal HLA-DQA1 protein (recombinant peptides to be produced by commercial organizations based on published consensus sequences) in agarose gels by immunofixation. This is because of the high degree of homology between the two proteins.

a. HLA-DQA2-specific antibodies are tested in agarose gels against samples of concentrated supernatants from papain digests or nonionic detergent extracts of B cell lines containing class II molecules from 5 low and 5 high-expressing controls and 5 T1D patients. Only antibodies producing single bands in these reactions and with recombinant HLA-DQA2 but not HLA-DQA1 are used.

b. These antibodies are used to quantitate HLA-DQA2 on the B cell or the B cell line cell surface from patients and from normal subjects by flow cytometry, using fluorescently labeled anti-mouse Ig. B cell lines or B cells are negatively selected from peripheral blood mononuclear cells, using antibodies to non-B cell surface antigens such as anti-CD14, anti-CD2, anti-CD3 and anti-CD16. Alternatively, peripheral blood mononuclear cells are examined directly and two antibodies, one directed against HLA-DQA2, the other against a B cell marker such as CD19 or CD20, labeled with different fluors, are used. Expression of HLA-DQA2 in T1D patients or normal hyperexpressers by cells other than B cells (CD19; CD20, HLA-DQA2⁺), particularly T cells (CD3⁺), are ascertained to define other alterations accompanying upregulation. Differences in mean fluorescence intensity or in the percent-positive B cells is ascertained to determine whether the difference in levels of HLA-DQA2 is the result of increased synthesis per cell or recruitment of more B cells or both. Higher HLA-DQA2 protein levels on T1D patient B cells (n=25) than controls (n=25) is correlated with higher transcription rates. Because smaller amounts of blood are required for flow cytometry than for the measurement of transcription rates, and assays are simpler, success with these methods is important for the development of clinical tests for young children at high risk for developing T1D.

c. Examine whether HLA-DQA2 exists as a monomer, a homodimer or a heterodimer in 25 normal subjects with normal expression, 25 normal subjects with increased expression and 25 T1D patients, to learn whether increased expression per se or increased expression in T1D is associated with differences from normal in the molecular forms of HLA-DQA2. This may also suggest mechanisms by which increased HLA-DAA2 expression leads to disease. Explore the possibilities of heterodimer formation of HLA-DQA2 not only with normally expressed HLA-DQB1, but also the normally non-expressed HLA-DQB2 or HLA-DQB3 molecules.

3. Complex formation involving HLA-DQA2. Anti-HLA-DQA2 is used in immunofixation after electrophoresis in 1% agarose gels and in western blotting after polyacrylamide gel electrophoresis with and without reducing reagent or SDS to identify homodimers and hetrodimers with various class II beta chains (HLA-DRB1, -DQB1, and -DPB1). If there is detectable transcription of either HLA-DQB2 or HLA-DQB3, recombinant proteins for the production of specific monoclonal antibodies are synthesized. The latter is used to detect hetrodimers with HLA-DQA2 after electrophoresis in agarose and polyacrylamide gel electrophoresis, as above.
4. Methylation in relation to susceptibility/protection-distinguishing SNPs in the first intron of HLA-DQA2. DNA methylation is the best-documented epigenetic change affecting gene expression, serving as a switch to activate or repress gene transcription. Using methylation-specific PCR to test distinguishing SNPs, beginning with the A/G SNP at position 1217 (see Table 2). Test 5 HLA-DQA2*S1S1 and 5 HLA-DQA2*S2S2 each among T1D patients, normal hyperexpressers and control normal expressers of HLA-DQA2. Umethylated human sperm DNA and sperm DNA methylated in vitro (New England Biolabs, Beverly, Mass.) serve as controls.

### Example 4

### Purpose

To determine whether B cell surface HLA-DQA2 assessed by flow cytometry strictly correlates with HLA-DQA2 transcription in T1D patients and normal adult controls.

### Rationale:

It is the main goal to improve the ability to detect impending T1D in high-risk sibs of patients with respect to sensitivity, specificity and potentially earlier detection than is now possible. We hope to utilize our findings with respect to the identification of HLA-DQA2 as the T1D MHC susceptibility gene and its hyperexpression in patients toward this end. The first task is to develop tests that are practical in very young subjects by virtue of small blood sample volume. This is accomplished by comparing transcription rates of HLA-DQA2 and cell surface levels of HLA-DQA2 in B cells and PBMC from subjects 15 years of age and older with both high and low levels of expression to determine whether both high transcription and high translation correlate with each other and with the presence or absence of T1D.

### Experimental Design:

a. Transcription rates of HLA-DQA2 in B cells from T1D patients and controls. B cells are known to constitutively express MHC class II proteins, independent of the MHC class II transactivator. Examine the expression of HLA-DQA2 in B cells and B cell lines (determining first in more subjects that rates are the same in freshly isolated B cells from 50 mL of whole blood, B cells in PBMC and in B cell lines from the same individual and stable over time). Quantitative RT-PCR is used to determine rates of transcription of HLA-DQA2 in B cells from T1D patients and age and sex-matched controls. Quantitative RT-PCR is performed with SYBR Ex Taq HS from Takara Mirus Bio (Madison, Wis.). SYBR Green I fluorescent dye is a sequence-independent, universal qRT-PCR detection reagent with the ability to bind to all dsDNA molecules. Real-time PCR primers are designed to assure maximal efficiency and sensitivity based on primer-3 and Olignucleotide Properties Calculator programs. Real-time PCR is performed in the iCycler iQ PCR detection system (Bio-Rad, Hercules, Calif.) using the following program: 10 sec of pre-incubation at 95° C. followed by 40 cycles for 5 sec at 95° C. and 30 sec at 60° C. Individual real-time PCR reactions are carried out in 20 uL volumes in a 96-well plate containing 1×SYBR Premix ExTaq, 0.2 uM forward primer, 0.2 uM reverse primer and varying amounts of template cDNA as described by the manufacturer (Takara Mirus Bio). GAPDH expression provides the internal standard. Avoid amplification of the highly homologous HLA-DQA1. Specific forward and reverse primers for unique HLA-DQA2 and HLA-DQA1 consensus exons (avoiding allelic differences) for these experiments are obtained. Controls include RT-PCR of HLA-DRA, HLA-DRB1, HLA-DQA1 and HLA-DQB1 in the same samples, as well. Study HLA-DQA2 expression in a total of 100 Caucasian patients over the age of 15 years with T1D, 100 high-risk sibs and 100-age and gender-matched Caucasian controls. This should provide a baseline of a wide variety of expression levels with which to compare cell surface levels of HLA-DQA2 in both B cells and PBMC. Since the results for expression so far (FIG. 1) indicate an order of magnitude difference in mean expression and results differ significantly (p<0.01) with only 10 patients and 13 controls, analysis of the number of subjects specified is of sufficient power, even is some fraction (we predict 11%) of normal subjects has high levels.

b. Transcription rates of HLA-DQA2 in B cells and peripheral blood mononuclear cells from T1D patients and random unrelated control individuals homozygous for HLA-DQA2 S and P alleles. An important questions to be answered is whether there are differences in mean transcription levels among HLA-DQA2*S1 S1, S1S2 and S2S2 patients or healthy individuals. Twenty-four such persons and 24 persons with 1 or 2 P alleles are tested for HLA-DQA2 expression and appropriately quantified. If the high expression rate in HLA-DQA2*S1S1 patients and controls is 60% and that of HLA-DQA2*S2S2 patients and controls is 20%, so that the average is 40% (if their frequencies are equal), 23 subjects each are required at alpha=0.05 and power=0.80. HLA-DQA2*P allele heterozygotes or homozygotes are not expected to have high expression. Even at 20% high expression by HLA-DQA2*P heterozygotes and 80% by HLA-DQA2*S homozygotes, only 10 samples each are required to show a significant difference at alpha=0.05 and power=0.80.

c. Levels of HLA-DQA2 protein on the surface of B cells. With no commercially available antibody to HLA-DQA2 protein, custom-made rabbit polyclonal antibodies to a recombinant HLA-DQA2 peptide is obtained. This is chosen on the basis of differences in coding DNA from HLA-DQA1 or its common alleles. Appropriate DNA is expressed in a suitable expression vector and the resulting peptide is purified and used for immunization as is or as a fusion protein. Antibodies are selected that react strongly with HLA-DQA2 protein. If there is some reactivity with recombinant normal HLA-DQA1 protein (recombinant proteins to be produced by commercial organizations based on published sequences) because of the high degree of homology between the two proteins, absorption with immobilized HLADQA1 is carried out. Only antibodies that are specific for HLA-DQA2 and potent are utilized. These antibodies are used to quantitate HLA-DQA2 on B cells or B cell lines and PBMC from patients and from normal subjects by flow cytometry, using fluorescently labels anti-rabbit Ig. B cells are either negatively selected from PBMC, using antibodies to non-B cell surface antigens, or positively selected with anti-CD19 and/or —CD20. Up to 100 patients with T1D and 100 controls are studied for whom expression levels are determined above. Levels of surface expression are correlated with mRNA levels in same cells. It is expected that the levels of HLA-DQA2 protein on B cells is proportional to transcription expression levels in donors with high expression, with the same number of positive B cells as in low expressers but ten-fold higher mean fluorescent intensity (MFI). It is also possible that MFI is the same but the number of positive B cells is increased ten-fold or there is a combination of the two mechanisms.

d. Levels of HLA-DQA2 protein on the surface of PBMC. Alternatively, analyze PBMC and utilize an antibody to a marker for B cells (CD19 or CD 20) and anti-HLA-DQA2 labeled with a different fluor. Determine if any cells beside B cells are positive for HLA-DQA2. Differences in MFI or in the percent positive B cells is ascertained to determine whether the difference in levels of HLA-DQA2 is the result of increased synthesis per cell or recruitment of more B cells, as above. In either case, HLA-DQA2 protein production by T1 patient B cells over most controls should correlate with higher transcription rates. As an alternative to assays for elevated gene transcription rates, flow cytometry offers advantages over qRT-PCR. It is simpler and requires smaller volumes of blood, which is of importance in the testing of young children.

### Example 5

### Purpose

To determine the time course of the selective increase in HLA-DQA2 expression by B cells from birth to late childhood.

### Rationale:

First confirm the association of HLA-DQA2 high expression with homozygosity for S alleles. Then address the basic question of whether the tenfold higher expression of HLA-DQA2 of some susceptibility allele homozygotes occurs early in fetal development (i.e. before birth) or at some time during childhood. Toward this end, determine HLA-DQA2 expression in 100 umbilical cord samples (to be obtained from the MGH) and at six-month intervals in high risk sibs of patients.

### Experimental Design:

a. HLA-DQA2 levels in siblings of T1D patients. In order to better understand the relationship of altered expression of HLA-DQA2 to the P and S alleles and to T1D, studies in families with known MHC haplotypes and a T1D-affected member are conducted. A critical question is whether there are healthy members who are homozygous for S alleles who also have elevated levels of HLA-DQA2 transcription but no T1D because they lack complete non-MHC susceptibility. Healthy members are tested for autoantibodies (see Example 6). To the extent possible, based on results from Example 4, tests are conducted using flow cytometry. 100 sibs of T1D patients who are homozygous for HLA-DQA2 susceptibility alleles and 100 who are not are tested for expression levels of HLA-DQA2. Another 100 healthy age-, gender-, and ethnically-matched control subjects (in addition to those tested in Example 4) are tested to match the number of healthy sibs tested. Siblings from a wide variety of ages (newborn to post-adolescent) are enrolled, but at least 10% who are newborn to 2 years of age and at least 50% who are pre-adolescent are not included. Families who are willing to return to provide a blood sample up to twice a year are recruited.

b. Transcription rates of HLA-DQA2 in B cells from members of families with more than one T1D patient. As a subset of the subjects enrolled from the studies in SA2 (a), up to 30 multiplex families are studied, 10 with MHC-identical sibs, 10 with haploidentical sibs and up to 10 with non-MHC-sharing sibs. It is expected that all patients are HLA-DQA2*SS and are high expressers. Some family members who are HLA-DQA2*S allele homozygotes may be high expressers but healthy because they have not inherited all of the non-MHC T1D susceptibility genes. Other HLA-DQA2**SS family members are low expressers and healthy. It is also expected that the parents of most MHC-identical affected sibs are HLA-DQA2*S heterozygotes, whereas at least one parent of the haploidentical sibs is a homozygote, as are both parents of the non-sharing sib.

c. Longitudinal analysis of cell surface levels of HLA-DQA2 in healthy relatives of patients with T1D. If HLA-DQA2 is the susceptibility locus, determining homozygosity for susceptibility alleles to define MHC susceptibility for T1D has a considerable advantage over current class II typing. If some HLA-DQA2*S allele healthy homozygotes in the T1D families have elevated transcription levels (predicted rate to be about 40% or the same as the concordance rate for T1D in monozygotic twins and 0 in P allele heterozygotes or homozygotes), stored or freshly obtained PBMC or B cells from non-T1D-affected MHC-typed persons who carry two T1D susceptibility-conferring CEHs are tested. These subjects include both siblings of patients with T1D and controls with no family history of T1D. HLA-DQA2 transcription in susceptibility gene homozygous sibs of patients is studied longitudinally to determine when increased expression is first detectable. If this is detectable at the predicted maximal rate (about 40%) in newborns, the studies are extended to fetal expression (although this may not be feasible) by isolating cDNA from cells of amniotic fluid and using low cell number RT-PCR developed in the laboratory. Samples obtained every 6 to 12 months from these subjects are tested and monitored for changes in the frequency of B (or T) cells positive for HLA-DQA2 as well as the protein's cell surface levels (MFI). Although HLA-DQA2 levels do not change with time, it is useful to know whether high expression is fixed at birth or it somehow temporally related to overt conversion of T1D (see Example 6).

### Example 6

### Purpose

To determine if identification of homozygosity for HLA-DQA2 susceptibility alleles and increased HLA-DQA2 expression improves predictability of impending T1D in high-risk subjects and if altered expression of HLA-DQA2 precedes the detection of autoantibodies in impending T1D.

### Rationale:

The overall goal is this research is to improve the detection of impending T1D in the high-risk sibs of T1D patients. Determine whether typing susceptibility alleles of the MHC susceptibility locus HLA-DQA2 and determination of hyperexpression of HLA-DQA2 improves the ability to predict and/or provides earlier evidence for impending T1D than current methods utilizing authoantibody testing with or without HLA-DRB1, -DQA1 and -DQB1 typing. Determining homozygosity for HLA-DQA2*S1 or *S2 or double heterozygosity (*S1S2) for defining MHC susceptibility for T1D has a considerable advantage. HLA-DQA2 typing should be 100% accurate in identifying MHC susceptibles. On the other hand, relying on “high risk” HLA-DR, -DQ haplotypes fails to detect many potential future victims of the disease.

### Experimental Design:

a. Determining T1D susceptibility by HLA-DQA2 genotyping. All 300 subjects enrolled for expression studies in Examples 4 and 5 (100 T1D subjects, 100 sibs and 100 healthy controls with no family history of T1D) are allele typed for HLA-DQA2 as well as HLA-DRB1 and HLA-DQB1. HLA-DQA2 alleles can be typed using 3 separate PCR reactions followed by sequence analysis. To the extent necessary to assign haplotypes unambiguously, first-degree relatives of these subjects is genotyped for the same loci. Susceptibility to T1D based on homozygosity for S alleles at HLA-DQA2 is defined. HLA-DQA2*S alleles are defined by those HLA-DQA2 alleles found in T1D subjects with “high-risk” HLA-DR, -DQ haplotypes.

b. Using increased HLA-DQA2 transcription to improve the prediction of impending T1D in high-risk individuals. All healthy subjects enrolled in the study are tested for IAA, IA-2 and GADA autoantibodies. HLA-DQA2 transcription is studied in susceptibility gene homozygous sibs of patients longitudinally to determine when increased expression if first detectable. Determine whether the addition of HLA-DQA2 expressions assay to autoantibody tests improves the ability to predict impending T1D. The 200 sibs of T1D patients described in SA2 is studied at 6-month intervals. Healthy persons homozygous for susceptibility alleles (we estimate over a quarter of the population) unrelated to a patient with T1D but with elevated HLA-DQA2 expression (11% of normal subjects detected in Examples 4 and 5 plus others available) are tested for autoantibodies for a total of 100 normal HLA-DQA2*SS subjects.

c. Comparison of results of analysis for HLA-DQA2 SNP typing, transcription and B cell surface protein carried out with results of HLA-DR, DQ typing and levels of autoantibodies. Sequential analyses (from birth whenever possible) of HLA-DQA2 expression in HLA-DQA2 SNP-typed high-risk sibs of T1D patients are compared with levels of autoantibodies to determine which appear first and better predict the onset of T1D. Any difference in time of earlier appearance are tested for significance. The 100 sibs of T1D patients described in SA2 are studied.

### Results:

If a small percentage of HLA-DQA2*PS individuals with or without T1D have increased expression, it may be that upregulation is not absolutely HLA-DQA2*S allele determined. If there are any exceptions to HLA-DQA2 hyperexpression in T1D, re-examine the basis for the diagnosis of T1D. If the diagnosis of T1D is clear, search out alternative mechanisms in such patients on the possibility that this represents genetic heterogeneity. Similarly, if significantly fewer than 40% of normal HLA-DQA2 susceptibility allele homozygotes show hyperexpression, it may have to do with a difference in HLA-DQA2 *S1 to *S2 expression ratio in normals compared with patients. This should be detectable by separately measuring the rates of expression in T1D and normal homozygous for *S1 and *S2 as well as in *S1S2 heterozygotes. If an anti-DQA2 antibody is reactive with normal but not patient HLA-DQA2, this suggests conformational change in patient HLA-DQA2 with loss of a normal epitope, provided that patient HLA-DQA2 reacts with antibodies that do not distinguish between normal and patients HLA-DQA2 protein. If this occurs, use anti-HLA-DQA2 that reacts with patient as well as normal HLA-DQA2 to isolate the patient protein for immunization to produce a patient-specific HLA-DQA2 for further analysis.

Until it is shown that the nucleotides in the SNP haplotypes defined as HLA-DQA2*S alleles above are chemically or otherwise modified in upregulated genes compared with those with lower expression, it is necessary to deal with markers, albeit intragenic markers. If a chemical modification in any first intron SNP is not shown, the 5′UT region is examined. If such change in either region is not shown, consider some previously unknown chemical change or that the change in expression is related to siRNA binding sites or other RNA or protein-mediated regulatory mechanisms affecting HLA-DQA2*S and *P alleles differentially.

If the expression of HLA-DQA2 is increased in patients with T1D and the molecular and functional interactions of HLA-DQA2 are altered in patients with T1D, the case that this gene is the MHC susceptibility gene for T1D is considerably strengthened. Given that no susceptibility gene in any complex (polygenic) disease has been definitely identified, establishing strict criteria is important. If only HLA-DQA2 among class II genes is affected, this suggests a causal role, as does hyperexpression in some normal subjects. If the changes in HLA-DQA2 expression precede autoantibody markers of impending T1D in high-risk individuals, this also suggests a causal role for these changes and reinforces the identity of HLA-DQA2 as the susceptibility locus. Finally, if the rate of high expression in normal homozygotes for HLA-DQA2 susceptibility alleles is about 40%, this further suggests a casual role in T1D rather than T1D being the cause of the increased HLA-DQA2 expression.

Positive results in the present study spurs investigations of the relationship between disease and non-coding nucleotide differences and changes in gene expression and gene product conformation of candidate genes. If changes in methylation or acetylation or the like in the critical regions of non-coding sequences are found to correlate with the presence of T1D and selective hyperexpression, possible factors influencing these chemical changes are investigated. Although this is made less likely by the predicted results, any environmental influences on penetrance can be studied at the molecular level in tissue culture. If conformational differences from healthy controls are found in the HLA-DQA2 protein in T1D patients, this can spur an investigation into the functions of the “new” forms and their relation to T1D.

The experiments described herein may produce evidence of complex formation but no reaction with antibodies to HLA-DQB1. The HLA-DQB2 and HLA-DQB3 genes are considered to be pseudogenes because no expression is detected in normal subjects, despite the absence of premature stop codons or other structural reasons for lack of expression. Since HLA-DQB2 and HLA-DQB3 are tested for transcription and it is planned, if there is transcription, to develop antibodies to recombinant HLA-DQB2 and HLA-DQB3 proteins, it can be determined if the corresponding genes are, in fact, translated in T1D patients and high normal expressers and one or another forms complexes with HLA-DQA2.

The application of positive findings produces clinical tests for HLa-DQA2*S and *P allele typing and increased HLA-DQA2 gene expression and altered protein product to add to tests to predict disease in high-risk relative of patients with T1D, particularly if abnormalities precede the appearance of autoantibodies. It is well-established that only 55% of siblings of patients with T1D who are destined to develop T1D themselves are MHC-identical to the patients. The present studies can increase the accuracy of prediction on the basis of MHC susceptibility allele homozygosity and the presence of hyperexpression. Another exceedingly important practical results is the search for pharmacologic agents that might influence the chemical change in non-coding DNA associated with and perhaps responsible for changes in expression. This can prevent disease onset in high-risk subjects or even reverse existing T1D.

### Example 7

Patients selected are ketosis-prone, insulin-dependent since diagnosis and presented before age 30. Mean onset of diabetes is 14.8 years (n=11; SD=7.4 years) and patients tested (n=12) are GAD65 and ICA512 autoantibody-positive. All subjects give informed consent. Genomic DNA is obtained from peripheral blood mononuclear cells, EDTA-treated whole blood or lymphoblastoid cell lines is isolated using the QIAamp DNA mini kit (Qiagen, Valencia, Calif.). Patients carry HLA-DQB1*0602 as determined by direct sequencing of group-specific second exon class II amplicons. Other typing is as described previously.

HLA-DQA2 first itron SNPs distinguishing the [HLA-B*0701, DRB1*1501, DQB1*0602] CEH from other haplotypes determined by the Sanger Centre are analyzed. Comparable sequences in a T1D patient homozygous for the [HLA-B60/62, SB42, DRB1*0401, DQB1*0302] CEH are determined. HLA-DQA2 SNPs (and the corresponding refSNP IDs) are at positions 899 (rs5018343), 1150 (rs9276408), 1157 (rs9276409), 1176 (rs9276410) and 3446 (rs9276434). Patients homozygous for SNPs not found in consensus normal HLA-DRB1*1501, -DQB1*0602 CEHs defined crossingover.

Eleven HLA-DQB1*0602-bearing haplotypes in 6 unrelated normal homozygotes established phase. Phase is determined in heterozygotes through SNPs of known CEHs when they are the other haplotypes or by pedigree analysis.

| | |
|---|---|
| Primers used are: | |
| F1: | |
| 5′-AAATGGGCATGTAGATATGCTTT-3′; | (SEQ ID NO: 17) |
| R1: | |
| 5′-TTCCAGTTCACTTCCCCTTTT-3′; | (SEQ ID NO. 18) |
| R1a: | |
| 5′TCAGAATGGGAGCAGCTATG-3′; | (SEQ ID NO. 19) |
| F2 | |
| 5′-CAGGATCCATCTCTGACTCC-3′; | (SEQ ID NO. 20) |
| R2: | |
| 5′-TGCCCACACTGTTCCTAATGG-3′; | (SEQ ID NO. 21) |
| F3: | |
| 5′-CGTTCCTACATGCTTTTCTTTG-3′; | (SEQ ID NO. 22) |
| and | |
| R3: | |
| 5′-GGCACAGTCCCTGAACAATC-3′. | (SEQ ID NO. 23) |

PCR reactions (50 uL) use AmpliTaq DNA polymerase (2.5 U), MgCl₂ (1.5 mM), and dNTP (0.4 mM) with: 1 cycle at 94° C. (2 min); followed by 35 cycles at 94° C. (15s), 60° C. (30s), 68° C. (2 min) with a final extension at 72° C. for 10 min. The annealing cycle is 62° C. (30s) for the PCR reactions 1F/1Ra and 2F/2R. Ethidium bromide-stained PCR products are excised from agarose gels, purified using the QUIAEX II gel extraction kit (Qiagen, Valencia, Calif.) and sequenced by dideoxy sequencing using the Big Dye Terminator V3.0 chemistry (Davis Sequencing, Davis, Calif.). Sequences are compared using alignment software and the visual inspection of chromatograms.

### Results:

Table 5 presents 20 independent instances of HLA-DQA2 first intron SNP haplotypes of normal unrelated healthy subjects who carry HLA-DQB1*0602-bearing CEHs. Nineteen of 20 or 95% of the independent normal examples of CEHs with HLA-DQB1*0602 are identical and fixed in the first intron of HLA-DQA2. A single individual (MOB) has 3 aberrant SNPs, at positions 1150, 1157 and 3446, suggesting an ancient crossover on 1 of the HLA-DQB1*0602-bearing haplotypes. Therefore, the DNA fixity that characterizes independent examples of the CEHs with HLA-DQB1*0602 in these subjects extends, in general, through HLA-DQA2, since virtually all independent examples have the same SNP haplotypes.

Analyses of the same SNPs in the T1D patients who carry the normally protective HLA-DQB1*0602 haplotype are shown in Table 6. One patient has no SNP in common with the consensus haplotypes, 1 has only 1 in common and 1 has 2 in common. In 6 patients, there are 2 SNPs that differ from the consensus normal haplotypes and in 6 other patients there is only 1 aberrant SNP. Thus, in the majority of patients with informative SNPs (9 of 15), there are 2 or more SNPs that differ from the consensus normal HLA-DQA2 first intron SNP haplotypes on HLA-DQB1*0602-bearing CEHs.

TABLE 5
HLA-DQA2 first intron SNPs on 20 independent
normal haplotypes containing HLA-DQB1*0602

| | Nucleotide position^{a} | | | | |
|---|---|---|---|---|---|
| Donor | 899 | 1150 | 1157 | 1176 | 3446 |
| 1 (PGF)^{b} | A | 1150 | 1157 | 1176 | 3446 |
| 2 (RCH a) | A | T | T | G | C |
| 3 (RCH b) | A | T | T | G | C |
| 4 (BHU a) | A | T | T | G | C |
| 5 (BHU b) | A | T | T | G | C |
| 6 (LD2B a) | A | T | T | G | C |
| 7 (LD2B b) | A | T | T | G | C |
| 8 (MOB a) | A | T | T | G | C |
| 9 (MOB b) | A | G | C | G | T |
| 10 (CCO)^{c} | A | T | T | G | C |
| 11 (KBO)^{d} | A | T | T | G | C |
| 12 (SBR)^{d} | A | T | T | G | C |
| 13 (JBO)^{d} | A | T | T | G | C |
| 14 (DKE)^{d} | A | T | T | G | C |
| 15 (RBR)^{d} | A | T | T | G | C |
| 16 (JFL)^{d} | A | T | T | G | C |
| 17 (RBO)^{d} | A | T | T | G | C |
| 18 (JHI)^{d} | A | T | T | G | C |
| 19 (kaBO)^{d} | A | T | T | G | C |
| 20 (JOR)^{d} | A | T | T | G | C |

^{a}Nucleotide position from PGF transcription start site.
^{b}PGF is from the Sanger Centre sequence [37].
^{c}Includes the normal HLA-DQA2 SNP haplotypes of CCO in Table 3.
^{d}Normal HLA-DQA2 SNP hapolotype from phase determination.

None of the informative patients (1-15) have the SNP haplotypes that characterizes the HLA-DQA2 genes on the [HLA-B7, DR2, DQB1*0602] CEH in healthy subjects, as shown in Table 5. Four patients (16-19) were heterozygous at all 5 nucleotide positions and therefore uninformative for identity or non-identity with the consensus SNP haplotypes since no first-degree relative was available to assign phase. The results indicate that another locus centromeric to HLA-DQB1 is a major determinant of genetic protection from and susceptibility to T1D associated with HLA-DQB1*0602 haplotypes, suggesting this is the true major susceptibility locus within the MHC. Since HLA-DQA2 is the gene analyzed in all patients, the true MHC T1D susceptibility locus is HLA-DQA2 or at least a locus centromeric to HLA-DQB1.

TABLE 6
HLA-DQA2 first itron SNP's in PGF and 19 TID patients who carry
HLA-DQB1*0602

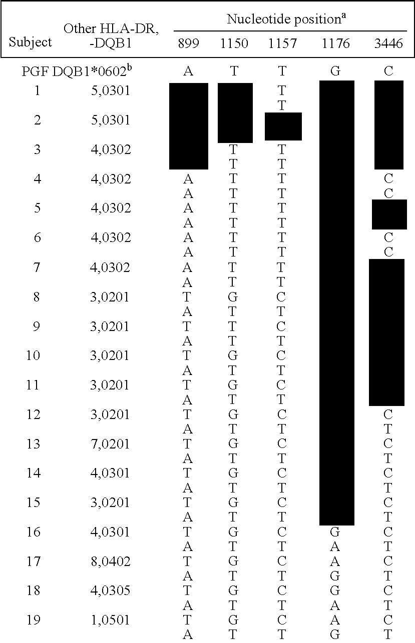

^{a}Nucleotide position from PGF transcription start site.
^{b}Normal HLA-DQB1*0602. Homozygosity discordant with PGF sequence highlighted in grey

A number of embodiments of the inventions have been described herein. Nevertheless, it will be understood that various modifications may be made to the invention without departing from its spirit and scope. Accordingly, embodiments other than those specifically described herein are intended to be embraced by the following claims. Those skilled in the art will be able to ascertain, using no more than routine experimentation, many equivalents of the specific embodiments of the invention described herein. These and all other equivalents are intended to be encompassed by the following claims.

What is claimed is:

1. A diagnostic method for determining the predisposition of an individual for developing type 1 diabetes, said method comprising the steps of
measuring the level of transcription or translation of the HLA-DQA2 gene in the subject individual,
comparing the level of transcription of translation against a known standard for a control individual and
determining, based on the degree or increase of the transcription or translation level for the subject individual against the control, whether the subject individual has a predisposition for developing type 1 diabetes.

2. The diagnostic method of claim 1 wherein the level of protein expressed by the HLA DQA2 gene is measured and used for the diagnosis of type 1 diabetes.

3. The diagnostic method of claim 2 wherein the level of protein expression is measured using an antibody to the protein.

4. The diagnostic method of claim 2 wherein the protein is located on the surface of a B cell.

5. The diagnostic method of claim 1 wherein a level of increase of at least 10 fold is used as an indication of a predisposition to develop type 1 diabetes.

6. The diagnostic method of claim 1 which is conducted in vitro.

7. A method for treating a subject having, or at risk of developing, type 1 diabetes comprising administering to the subject an effective amount of a composition comprising an inhibitor for HLA-DQA2 transcription or translation.

8. The method of claim 7 wherein the inhibitor comprises an antibody to a protein produced by HLA-DQA2.

9. The method of claim 8 wherein the protein is on the surface of a B cell.

10. The method of claim 8 wherein the antibody is a monoclonal antibody.

11. The method of claim 7 wherein the inhibitor interferes with or blocks the transcription of HLA-DQA2.

12. The method of claim 7 wherein the inhibitor interferes with or blocks the translation of HLA-DQA2.

13. The method of claim 7 wherein the inhibitor is an siRNA molecule which inhibits the synthesis, post-translational modification or functioning of HLA-DQA2.

14. A pharmaceutical composition for the treatment of type 1 diabetes comprising an HLA-DQA2 inhibitor, a pharmaceutically acceptable carrier, and an adjuvant.

15. The pharmaceutical composition of claim 14 which is an oral formulation.

16. A method for identifying a molecule for treating or diagnosing type 1 diabetes said method comprising contacting a molecule of interest with HLA-DQA2, fragments thereof, or a protein expressed by HLA-DQA2 on B cells, and measuring the inhibitory effect of the molecule of interest.

17. The method of claim 16 wherein the molecule of interest is part of a library of molecules.


SEQUENCE LISTING (23 sequences)

SEQ ID NO: 1 - DNA, 20 bp, Artificial Sequence
  pcr primer
cgaccactttgtcaagctca

SEQ ID NO: 2 - DNA, 20 bp, Artificial Sequence
  PCR primer
aggggtctacatggcaactg

SEQ ID NO: 3 - DNA, 19 bp, Artificial Sequence
  PCR primer
ggtgtaaacttgtaccagt

SEQ ID NO: 4 - DNA, 26 bp, Artificial Sequence
  PCR primer
ccttggtgtctggaagcaccaactga

SEQ ID NO: 5 - DNA, 21 bp, Artificial Sequence
  PCR primer
ctccagcatggtgtgtgtctg

SEQ ID NO: 6 - DNA, 18 bp, Artificial Sequence
  PCR primer
gctgggtctttgaaggat

SEQ ID NO: 7 - DNA, 17 bp, Artificial Sequence
  PCR primer
cgcttcgacagcgacgt

SEQ ID NO: 8 - DNA, 22 bp, Artificial Sequence
  PCR primer
ccacctcgtagttgtgtctgca

SEQ ID NO: 9 - DNA, 21 bp, Artificial Sequence
  PCR primer
gccaacctggaaatcatgaca

SEQ ID NO: 10 - DNA, 22 bp, Artificial Sequence
  PCR primer
tcaaagactagggatgtttcca

SEQ ID NO: 11 - DNA, 26 bp, Artificial Sequence
  PCR primer
cataaagcatatctacatgcccattt

SEQ ID NO: 12 - DNA, 23 bp, Artificial Sequence
  PCR primer
aaatgggcatgtagatatgcttt

SEQ ID NO: 13 - DNA, 21 bp, Artificial Sequence
  PCR primer
ttccagttcacttcccctttt

SEQ ID NO: 14 - DNA, 21 bp, Artificial Sequence
  PCR primer
tcagaatggggagcagctatg

SEQ ID NO: 15 - DNA, 21 bp, Artificial Sequence
  PCR primer
taaccatagctgctcccattc

SEQ ID NO: 16 - DNA, 20 bp, Artificial Sequence
  PCR primer
agctactcggggaggctgag

SEQ ID NO: 17 - DNA, 23 bp, Artificial Sequence
  PCR primer
aaatgggcatgtagatatgcttt

SEQ ID NO: 18 - DNA, 21 bp, Artificial Sequence
  PCR primer
ttccagttcacttcccctttt

SEQ ID NO: 19 - DNA, 20 bp, Artificial Sequence
  PCR primer
tcagaatgggagcagctatg

SEQ ID NO: 20 - DNA, 20 bp, Artificial Sequence
  PCR primer
caggatccatctctgactcc

SEQ ID NO: 21 - DNA, 21 bp, Artificial Sequence
  PCR primer
tgcccacactgttcctaatgg

SEQ ID NO: 22 - DNA, 22 bp, Artificial Sequence
  PCR primer
cgttcctacatgcttttctttg

SEQ ID NO: 23 - DNA, 20 bp, Artificial Sequence
  PCR primer
ggcacagtccctgaacaatc